(19) United States
(12) Patent Application Publication
(10) Pub. No.: US 2024/0417385 A1
(43) Pub. Date: Dec. 19, 2024

(54) COMPOUND AND USE THEREOF FOR TREATING EMOTIONAL BEHAVIORAL DISORDERS

(71) Applicant: NEUROVENTI INC., Seoul (KR)

(72) Inventors: CHAN YOUNG SHIN, Seoul (KR); KYOUNG JA KWON, Seoul (KR); CHILLY GAY REMONDE, Bukidnon (PH); SE JIN JEON, Seoul (KR); KYU SUK CHO, Seoul (KR); JUNG HYUN CHOI, Seoul (KR); EDSON LUCK GONZALES, Southern Leyte (PH); EUN YEONG SONG, Seoul (KR); SO HYUN JOO, Seoul (KR); SURESH PAUDEL, Tanahun (NP); KYEONG MAN KIM, Gwangju (KR); JAE HOON CHEONG, Jeollabuk-do (KR)

(21) Appl. No.: 18/690,418

(22) PCT Filed: Sep. 8, 2022
(86) PCT No.: PCT/KR2022/013519

§ 371 Date: Mar. 8, 2024

(30) Foreign Application Priority Data
Sep. 8, 2021 (KR) 10-2021-0119526
Sep. 8, 2022 (KR) 10-2022-0113977

(51) Int. Cl. C07D 401/12; A61K 31/496; A61P 25/00; C07D 295/215; C07D 405/12; C07D 409/12

(52) U.S. Cl. CPC C07D 401/12; A61K 31/496; A61P 25/00; C07D 295/215; C07D 405/12; C07D 409/12

(57) ABSTRACT

A compound is represented by Formula 1, a stereoisomer or a pharmaceutically acceptable salt thereof. The compound inhibits agmatinase and is expected to be helpful for improving autism spectrum disorder, emotional and behavioral disorder such as schizophrenia, as well as emotional and behavioral disorder-related symptoms such as lack of sociality, hyperactivity or the like.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2022/013519, filed Sep. 8, 2022, which claims priority to the benefit of Korean patent application Nos. 10-2021-0119526 filed on Sep. 8, 2021, and 10-2022-0113977 filed on Sep. 8, 2022, in the Korean intellectual property office, the entire contents of which are incorporated herein by reference.

### BACKGROUND

### 1. Technical Field

The present invention relates to a novel compound, and a use thereof for treating emotional and behavioral disorders.

### 2. Background Art

Emotional and behavioral disorders are the only mental disorders for which there is no cure, and it may be defined as a spectrum disease that exhibits difficulties in social and communication and stereotyped behaviors, and shows various coexisting symptoms such as seizures, anxiety, depression, aggression, cognitive impairment and the like.

The clear pathogenesis of emotional and behavioral disorders has not been identified, and the subject on which the most research has been conducted is the excitation/inhibition imbalance. By regulating the imbalance of excitatory/inhibitory neurons, it influences neural connectivity and integration, suggesting the possibility of improving core symptoms of emotional and behavioral disorders such as autism.

### SUMMARY

An object of the present invention is to provide a novel compound, a stereoisomer or a pharmaceutically acceptable salt thereof.

Another object of the present invention is to provide a novel compound useful for the prevention or treatment of emotional and behavioral disorders, a stereoisomer or a pharmaceutically acceptable salt thereof.

In addition, another object of the present invention is to provide a novel compound, a stereoisomer or a pharmaceutically acceptable salt thereof, which is useful for preventing or treating emotional and behavioral disorder-related symptoms.

Further, another object of the present invention is to provide a pharmaceutical composition for the prevention or treatment of emotional and behavioral disorders, which includes a novel compound, a stereoisomer or a pharmaceutically acceptable salt thereof.

1. A compound represented by Formula 1 below, a stereoisomer or a pharmaceutically acceptable salt thereof:

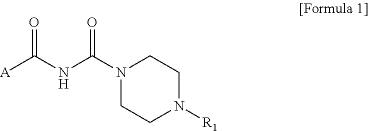

(wherein, R₁ is hydrogen or a tert-butyloxycarbonyl group,
A is a single or double cyclic group of C₅-C₁₀,
each ring of the cyclic group is unsubstituted or substituted with 1 to 3 heteroatoms, and
the cyclic group is unsubstituted or substituted with halogen, C₁-C₅ alkyl, C₁-C₅ alkoxy, CN or NO₂).

2. The compound, a stereoisomer or a pharmaceutically acceptable salt thereof according to the above 1, wherein A is selected from the group consisting of the following cyclic groups:

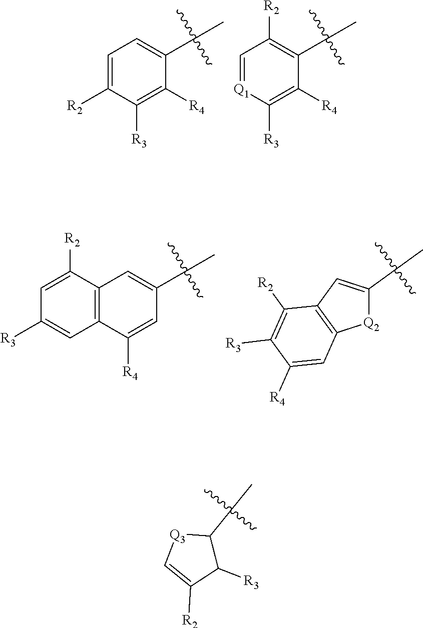

(wherein R₂, R₃ and R₄ are each independently hydrogen, halogen, C₁-C₅ alkyl, C₁-C₅ alkoxy, CN or NO₂, and
Q₁, Q₂ and Q₃ are each independently N, O or S).

3. The compound, a stereoisomer or a pharmaceutically acceptable salt thereof according to the above 1, wherein A is selected from the group consisting of the following cyclic groups:

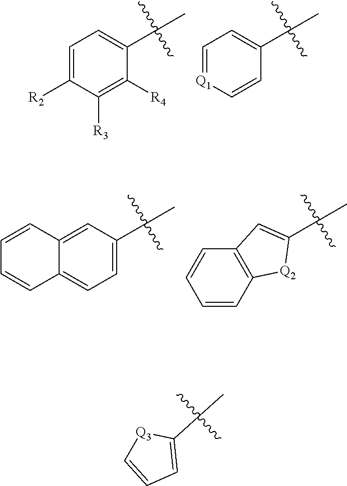

(wherein R₂, R₃ and R₄ are each independently hydrogen, halogen, C₁-C₅ alkyl, C₁-C₅ alkoxy, CN or NO₂; and
Q₁, Q₂ and Q₃ are each independently N, O or S).

4. The compound, a stereoisomer or a pharmaceutically acceptable salt thereof according to the above 1, wherein A is selected from the group consisting of the following cyclic groups:

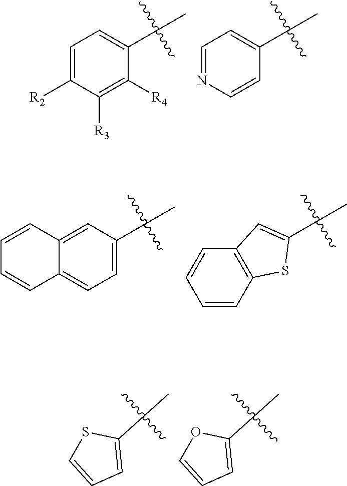

(wherein R₂, R₃ and R₄ are each independently hydrogen, halogen, C₁-C₅ alkyl, C₁-C₅ alkoxy, CN or NO₂).

5. The compound, a stereoisomer or a pharmaceutically acceptable salt thereof according to the above 1, wherein the compound is selected from the group consisting of the following compounds:
tert-butyl-4-((4-methylbenzoyl)carbamoyl)piperazine-1-carboxylate;
tert-butyl-4-((4-methoxybenzoyl)carbamoyl)piperazine-1-carboxylate;
tert-butyl-4-((4-chlorobenzoyl)carbamoyl)piperazine-1-carboxylate;
tert-butyl-4-(benzoylcarbamoyl)piperazine-1-carboxylate;
N-(4-methylbenzoyl)piperazine-1-carboxamide;
N-(4-methylbenzoyl)piperazine-1-carboxamide-2,2,2-trifluoroacetate;
N-(4-methoxybenzoyl)piperazine-1-carboxamide-2,2,2-trifluoroacetate;
N-(4-chlorobenzoyl)piperazine-1-carboxamide-2,2,2-trifluoroacetate;
N-benzoylpiperazine-1-carboxamide-2,2,2-trifluoroacetate;
N-(2-naphthoyl)piperazine-1-carboxamide-2,2,2-trifluoroacetate;
N-(4-cyanobenzoyl)piperazine-1-carboxamide-2,2,2-trifluoroacetate;
N-(furan-2-carbonyl)piperazine-1-carboxamide-2,2,2-trifluoroacetate;
N-(4-nitrobenzoyl)piperazine-1-carboxamide-2,2,2-trifluoroacetate;
N-(2-naphthoyl)piperazine-1-carboxamide hydrochloride;
N-(4-cyanobenzoyl)piperazine-1-carboxamide hydrochloride;
N-(4-nitrobenzoyl)piperazine-1-carboxamide hydrochloride;
N-benzoylpiperazine-1-carboxamide hydrochloride;
N-(furan-2-carbonyl)piperazine-1-carboxamide hydrochloride;
N-(4-methylbenzoyl)piperazine-1-carboxamide hydrochloride;
N-(4-methoxybenzoyl)piperazine-1-carboxamide hydrochloride;
N-(4-chlorobenzoyl)piperazine-1-carboxamide hydrochloride;
N-(2,4-dichlorobenzoyl)piperazine-1-carboxamide-2,2,2-trifluoroacetate;
N-(4-bromobenzoyl)piperazine-1-carboxamide-2,2,2-trifluoroacetate;
N-(thiophene-2-carbonyl)piperazine-1-carbxamide-2,2,2-trifluoroacetate;
N-(benzo[b]thiophene-2-carbonyl)piperazine-1-carboxamide-2,2,2-trifluoroacetate;
N-(4-fluorobenzoyl)piperazine-1-carboxamide-2,2,2-trifluoroacetate;
N-isonicotinoylpiperazine-1-carboxamide-2,2,2-trifluoroacetate;
N-(2,4-dichlorobenzoyl)piperazine-1-carboxamide hydrochloride;
N-(4-bromobenzoyl)piperazine-1-carboxamide hydrochloride;
N-(benzo[b]thiophene-2-carbonyl)piperazine-1-carboxamide hydrochloride;
N-isonicotinoylpiperazine-1-carboxamide hydrochloride;
N-(4-fluorobenzoyl)piperazine-1-carboxamide hydrochloride;
N-(thiophene-2-carbonyl)piperazine-1-carboxamide hydrochloride;
N-(3-chlorobenzoyl)piperazine-1-carboxamide hydrochloride;
N-(3-methylbenzoyl)piperazine-1-carboxamide hydrochloride;
N-(3-methoxybenzoyl)piperazine-1-carboxamide hydrochloride;
N-(3-chlorobenzoyl)piperazine-1-carboxamide-2,2,2-trifluoroacetate;
N-(3-methylbenzoyl)piperazine-1-carboxamide-2,2,2-trifluoroacetate; and
N-(3-methoxybenzoyl)piperazine-1-carboxamide-2,2,2-trifluoroacetate.

6. A pharmaceutical composition for preventing or treating emotional and behavioral disorder-related symptoms, the pharmaceutical composition including the compound, a stereoisomer or a pharmaceutically acceptable salt thereof as defined in any one of the above 1 to 5.

7. The pharmaceutical composition, wherein the above 6, wherein the emotional and behavioral disorder-related symptoms are any one selected from the group consisting of lack of sociability, lack of social cognitive intelligence, stereotyped behavior, hyperactivity, impulsivity and distraction.

8. A pharmaceutical composition for preventing or treating emotional and behavioral disorders, the pharmaceutical composition including the compound, a stereoisomer or a pharmaceutically acceptable salt thereof as defined in any one of the above 1 to 5.

9. A pharmaceutical composition for preventing or treating degenerative neurological diseases, the pharmaceutical composition including the compound, a stereoisomer or a pharmaceutically acceptable salt thereof as defined in any one of the above 1 to 5.

10. The pharmaceutical composition according to the above 8, wherein the emotional and behavioral disorder is any one selected from the group consisting of autism spectrum disorder, schizophrenia, obsessive-compulsive disorder, depression, anxiety disorder, panic disorder and attention deficit hyperactivity disorder.

The novel compound of the present invention, a stereoisomer or a pharmaceutically acceptable salt thereof acts as an agmatinase inhibitor and is effective in improving emotional and behavioral disorders, as well as symptoms related thereto.

### BRIEF DESCRIPTION OF THE DRAWINGS

FIG. 1 shows the agmatinase inhibitory activity of some of the compounds of the present invention shown in Tables 1 to 7.

FIG. 2 shows results confirming the social improvement effects of VPA mice by treatment with the compound of the present invention.

FIG. 3 shows results confirming the cognitive improvement effects of VPA mice by treatment with the compound of the present invention.

FIG. 4 shows a self-grooming test performed using a valproic acid (VPA)-induced an autism animal model.

FIGS. 5 and 6 show neuronal cell protective effects by treatment with the compound of the present invention.

### DETAILED DESCRIPTION

Hereinafter, the present invention will be described in detail.

All technical terms used in the present invention are used in the same meaning as those skilled in the art may generally understand in the related field of the present invention unless otherwise defined. Further, although preferred methods or samples will be described in the present specification, those similar or equivalent are also included in the scope of the present invention.

The present invention relates to a compound represented by Formula 1 below, a stereoisomer or a pharmaceutically acceptable salt thereof.

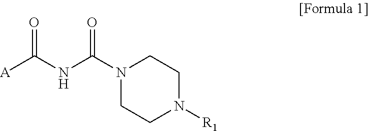

In the above formula, if any substituent is not indicated in a site although the site needs a substituent, it means that a hydrogen substituent is omitted, which would be applied to all formulae in the present invention.

In the above formula, R₁ is hydrogen or tert-butyloxycarbonyl group.

In the above formula, A is a single or double cyclic group of C₅-C₁₀, and may be, for example, benzene, naphthalene, indene, cyclopenta-1,3-diene, cyclohexane, tetrahydronaphthalene, etc., but it is limited thereto.

Each ring of the cyclic group may be substituted with 1 to 3 heteroatoms, and the heteroatoms refer to atoms capable of constituting a ring among atoms other than C. For example, 1 to 3 heteroatoms may be each independently substituted with any one or more selected from the group consisting of N, S and O, they are not limited thereto.

In the cyclic group, sites at which the heteroatom can be substituted may specifically be the same as Q₁ to Q₃ in the following listed structures, but they are not limited thereto.

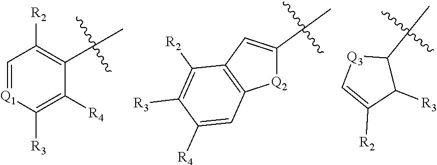

The cyclic group may be substituted with halogen, C₁-C₅ alkyl, C₁-C₅ alkoxy, CN or NO₂, and may be, for example, F, Cl, Br, methyl group (Me), ethyl group (Et), methoxy group (OMe), ethoxy group (OEt), CN, NO₂, and the like, but it is not limited thereto.

In the cyclic group, the sites at which the heteroatom can be substituted with halogen, C₁-C₅ alkyl, C₁-C₅ alkoxy, CN or NO₂ may be the same as R₂ to R₄, but they are not limited thereto.

According to one embodiment of the present invention, A may be selected from the following cyclic groups.

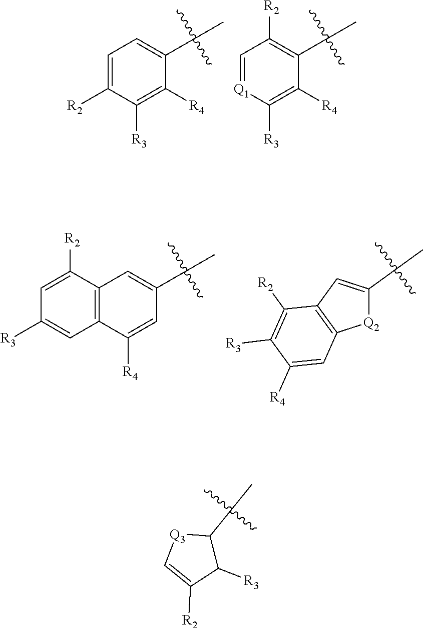

wherein R₂, R₃ and R₄ are each independently hydrogen, halogen, C₁-C₅ alkyl, C₁-C₅ alkoxy, CN or NO₂, and
Q₁, Q₂ and R₃ are each independently N, O or S).

In addition, according to one embodiment of the present invention, more specifically, A may be selected from the following cyclic groups.

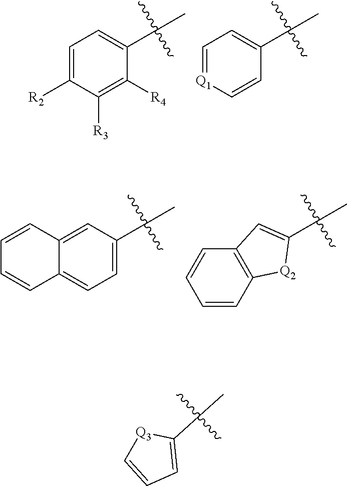

(wherein R₂, R₃ and R₄ are each independently hydrogen, halogen, C₁-C₅ alkyl, C₁-C₅ alkoxy, CN or NO₂, and
Q₁, Q₂ and Q₃ are each independently N, O or S).

Further, according to one embodiment of the present invention, most specifically, A may be selected from the following cyclic groups.

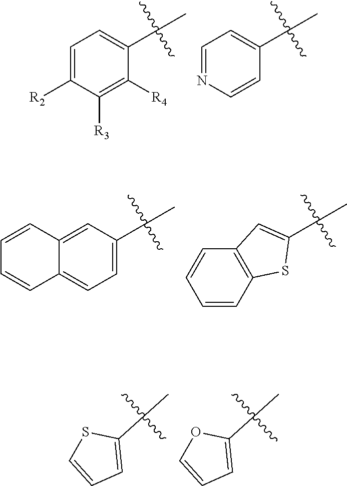

(Wherein R₂, R₃ and R₄ are each independently hydrogen, halogen, C₁-C₅ alkyl, C₁-C₅ alkoxy, CN or NO₂).

Tables 1 to 7 below show examples of the structure of the compound represented by Formula 1 through a combination of R₁ and A in the compound represented by Formula 1 and some of its pharmaceutically acceptable salts. The order of the following tables means the compound number of this specification.

TABLE 1

| No. | A | R₁ | R₂ | R₃ | R₄ | Q₁ | Q₂ | Q₃ | Salt |
|---|---|---|---|---|---|---|---|---|---|
| 1 | 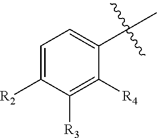 | Boc | Me | H | H | — | — | — | — |
| 2 | | Boc | Et | H | H | — | — | — | — |
| 3 | | Boc | OMe | H | H | — | — | — | — |
| 4 | | Boc | OEt | H | H | — | — | — | — |
| 5 | | Boc | Cl | H | H | — | — | — | — |
| 6 | | Boc | H | H | H | — | — | — | — |
| 7 | | Boc | NO₂ | H | H | — | — | — | — |
| 8 | | Boc | CN | H | H | — | — | — | — |
| 9 | | Boc | H | Me | H: | — | — | — | — |
| 10 | | Boc | H | Et | H | — | — | — | — |
| 11 | | Boc | H | OMe | H | — | — | — | — |
| 12 | | Boc | H | OEt | H | — | — | — | — |
| 13 | | Boc | H | Cl | H | — | — | — | — |
| 14 | | Boc | H | NO₂ | H | — | — | — | — |
| 15 | | Boc | H | H | Me | — | — | — | — |
| 16 | | Boc | H | H | OMe | — | — | — | — |
| 17 | | Boc | H | H | Cl | — | — | — | — |
| 18 | | Boc | H | H | NO₂ | — | — | — | — |
| 19 | | Boc | Me | Cl | H | — | — | — | — |
| 20 | | Boc | H | Me | Cl | — | — | — | — |
| 21 | | Boc | Me | H | Me | — | — | — | — |
| 22 | | Boc | Cl | Br | H | — | — | — | — |
| 23 | | Boc | NO₂ | Cl | H | — | — | — | — |
| 24 | | Boc | H | OMe | Br | — | — | — | — |
| 25 | | Boc | Me | OMe | H | — | — | — | — |
| 26 | | Boc | H | OMe | Br | — | — | — | — |
| 27 | | Boc | Me | H | Me | — | — | — | — |
| 28 | | H | Me | H | H | — | — | — | — |
| 29 | | H | Me | H | H | — | — | — | CF₃COOH |
| 30 | | H | Me | H | H | — | — | — | HCl |
| 31 | | H | Et | H | H | — | — | — | — |
| 32 | | H | OMe | H | H | — | — | — | CF₃COOH |
| 33 | | H | OMe | H | H | — | — | — | HCl |
| 34 | | H | OEt | H | H | — | — | — | — |
| 35 | | H | Cl | H | H | — | — | — | CF₃COOH |

TABLE 2

| No. | A | R₁ | R₂ | R₃ | R₄ | Q₁ | Q₂ | Q₃ | Salt |
|---|---|---|---|---|---|---|---|---|---|
| 36 | 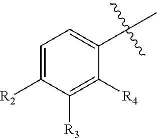 | H | Cl | H | H | — | — | — | HCl |
| 37 | | H | H | H | H | — | — | — | CF₃COOH |
| 38 | | H | H | H | H | — | — | — | HCl |
| 39 | | H | CN | H | H | — | — | — | CF₃COOH |
| 40 | | H | CN | H | H | — | — | — | HCl |
| 41 | | H | NO₂ | H | H | — | — | — | CF₃COOH |
| 42 | | H | NO₂ | H | H | — | — | — | HCl |
| 43 | | H | Cl | H | Cl | — | — | — | CF₃COOH |
| 44 | | H | Cl | H | Cl | — | — | — | HCl |
| 45 | | H | Br | H | H | — | — | — | CF₃COOH |
| 46 | | H | Br | H | H | — | — | — | HCl |
| 47 | | H | F | H | H | — | — | — | CF₃COOH |
| 48 | | H | F | H | H | — | — | — | HCl |
| 49 | | H | H | F | H | — | — | — | — |
| 50 | | H | H | Br | H | — | — | — | — |
| 51 | | H | H | Cl | H | — | — | — | HCl |
| 52 | | H | H | Cl | H | — | — | — | CF₃COOH |
| 53 | | H | H | Me | H | — | — | — | HCl |
| 54 | | H | H | Me | H | — | — | — | CH₃COOH |
| 55 | | H | H | OMe | H | — | — | — | HCl |
| 56 | | H | H | OMe | H | — | — | — | CF₃COOH |
| 57 | | H | Me | Br | H | — | — | — | — |
| 58 | | H | OMe | Cl | H | — | — | — | — |
| 59 | | H | H | Me | OMe | — | — | — | — |
| 60 | | H | Me | Cl | H | — | — | — | — |
| 61 | | H | H | Me | Cl | — | — | — | — |
| 62 | | H | Me | H | Me | — | — | — | — |
| 63 | | H | Cl | Br | H | — | — | — | — |
| 64 | | H | NO₂ | Cl | H | — | — | — | — |
| 65 | | H | H | OMe | Br | — | — | — | — |
| 66 | | H | Me | OMe | H | — | — | — | — |
| 67 | | H | H | OMe | Br | — | — | — | — |
| 68 | | H | H | H | Cl | — | — | — | — |
| 69 | | H | H | H | Br | — | — | — | — |
| 70 | | H | H | H | Me | — | — | — | — |
| 71 | | H | H | H | OMe | — | — | — | — |
| 72 | | H | H | H | CN | — | — | — | — |
| 73 | | H | H | OMe | CN | — | — | — | — |
| 74 | | H | H | Me | Br | — | — | — | — |
| 75 | | H | H | Me | Cl | — | — | — | — |

TABLE 3

| No. | A | R₁ | R₂ | R₃ | R₄ | Q₁ | Q₂ | Q₃ | Salt |
|---|---|---|---|---|---|---|---|---|---|
| 76 | 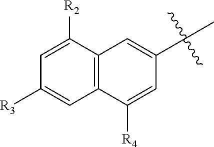 | H | H | H | H | — | — | — | CF₃COOH |
| 77 | | H | H | H | H | — | — | — | HCl |
| 78 | | H | Me | H | H | — | — | — | — |
| 79 | | H | Et | H | H | — | — | — | — |
| 80 | | H | OMe | H | H | — | — | — | — |
| 81 | | H | F | H | H | — | — | — | — |
| 82 | | H | Cl | H | H | — | — | — | — |
| 83 | | H | H | Me | H | — | — | — | — |
| 84 | | H | H | OMe | H | — | — | — | — |
| 85 | | H | H | Cl | H | — | — | — | — |
| 86 | | H | H | H | Me | — | — | — | — |
| 87 | | H | H | H | OMe | — | — | — | — |
| 88 | | H | H | H | Cl | — | — | — | — |
| 89 | | H | Cl | H | Cl | — | — | — | — |
| 90 | | H | Me | Cl | H | — | — | — | — |
| 91 | | H | OMe | Cl | H | — | — | — | — |
| 92 | | Boc | H | H | H | — | — | — | — |
| 93 | | Boc | Me | H | H | — | — | — | — |
| 94 | | Boc | Et | H | H | — | — | — | — |
| 95 | | Boc | OMe | H | H | — | — | — | — |
| 96 | | Boc | Cl | H | H | — | — | — | — |
| 97 | | Boc | H | Me | H | — | — | — | — |
| 98 | | Boc | H | OMe | H | — | — | — | — |
| 99 | | Boc | H | Cl | H | — | — | — | — |
| 100 | | Boc | H | H | Me | — | — | — | — |
| 101 | | Boc | H | H | OMe | — | — | — | — |

TABLE 4

| No. | A | R₁ | R₂ | R₃ | R₄ | Q₁ | Q₂ | Q₃ | Salt |
|---|---|---|---|---|---|---|---|---|---|
| 102 | 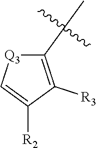 | H | H | H | — | — | — | O | CF₃COOH |
| 103 | | H | H | H | — | — | — | O | HCl |
| 104 | | H | H | H | — | — | — | S | CF₃COOH |
| 105 | | H | H | H | — | — | — | S | HCl |
| 108 | | H | H | H | — | — | — | N | — |
| 107 | | H | Me | H | — | — | — | O | — |
| 108 | | H | OMe | H | — | — | — | O | — |
| 109 | | H | Cl | H | — | — | — | O | — |
| 110 | | H | H | Me | — | — | — | O | — |
| 111 | | H | H | OMe | — | — | — | O | — |
| 112 | | H | H | CN | — | — | — | O | — |
| 113 | | H | Me | H | — | — | — | S | — |
| 114 | | H | OMe | H | — | — | — | S | — |
| 115 | | H | Cl | H | — | — | — | S | — |
| 116 | | H | H | Me | — | — | — | S | — |
| 117 | | H | H | OMe | — | — | — | S | — |
| 118 | | Boc | H | H | — | — | — | O | — |
| 119 | | Boc | H | H | — | — | — | S | — |
| 120 | | Boc | H | H | — | — | — | N | — |
| 121 | | Boc | Me | H | — | — | — | O | — |
| 122 | | Boc | OMe | H | — | — | — | O | — |
| 123 | | Boc | Cl | H | — | — | — | O | — |
| 124 | | Boc | Me | H | — | — | — | S | — |
| 125 | | Boc | OMe | H | — | — | — | S | — |
| 126 | | Boc | Cl | H | — | — | — | S | — |
| 127 | | Boc | H | Me | — | — | — | O | — |
| 128 | | Boc | H | Cl | — | — | — | O | — |

TABLE 5

| No. | A | R₁ | R₂ | R₃ | R₄ | Q₁ | Q₂ | Q₃ | Salt |
|---|---|---|---|---|---|---|---|---|---|
| 129 | 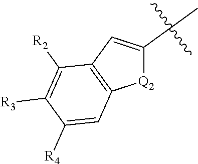 | H | H | H | H | — | S | — | CF₃OOOH |
| 130 | | H | H | H | H | — | S | — | HCl |
| 131 | | H | H | H | H | — | O | — | — |
| 132 | | H | H | H | H | — | N | — | — |
| 133 | | H | Me | H | H | — | S | — | — |
| 134 | | H | OMe | H | H | — | S | — | — |
| 135 | | H | Cl | H | H | — | S | — | — |
| 136 | | H | H | Me | H | — | S | — | — |
| 137 | | H | H | OMe | H | — | S | — | — |
| 138 | | H | H | CN | H | — | S | — | — |
| 139 | | H | H | H | Me | — | S | — | — |
| 140 | | H | H | H | OMe | — | S | — | — |
| 141 | | H | H | H | Cl | — | S | — | — |
| 142 | | Boc | H | H | H | — | S | — | — |
| 143 | | Boc | H | H | H | — | O | — | — |
| 144 | | Boc | H | H | H | — | N | — | — |
| 145 | | Boc | Me | H | H | — | S | — | — |
| 146 | | Boc | OMe | H | H | — | S | — | — |
| 147 | | Boc | Cl | H | H | — | S | — | — |
| 148 | | Boo | H | Me | H | — | S | — | — |
| 149 | | Boc | H | OMe | H | — | S | — | — |
| 150 | | Boc | H | CN | H | — | S | — | — |
| 151 | | Boc | H | H | Me | — | S | — | — |
| 152 | | Boc | H | H | OMe | — | S | — | — |
| 153 | | Boc | H | H | Cl | — | S | — | — |

TABLE 6

| No. | A | R₁ | R₂ | R₃ | R₄ | Q₁ | Q₂ | Q₃ | Salt |
|---|---|---|---|---|---|---|---|---|---|
| 154 | 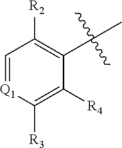 | H | H | H | H | N | — | — | CF₃COOH |
| 155 | | H | H | H | H | N | — | — | HCl |
| 156 | | H | H | H | H | O | — | — | — |
| 157 | | H | H | H | H | S | — | — | — |
| 158 | | H | Me | H | H | N | — | — | — |
| 159 | | H | Et | H | H | N | — | — | — |
| 160 | | H | OMe | H | H | N | — | — | — |
| 161 | | H | OEt | H | H | N | — | — | — |
| 162 | | H | Cl | H | H | N | — | — | — |
| 163 | | H | Me | H | H | O | — | — | — |
| 164 | | H | Et | H | H | O | — | — | — |
| 165 | | H | OMe | H | H | O | — | — | — |
| 166 | | H | OEt | H | H | O | — | — | — |
| 167 | | H | Cl | H | H | O | — | — | — |
| 168 | | H | Me | H | H | S | — | — | — |
| 169 | | H | Et | H | H | S | — | — | — |
| 170 | | H | OMe | H | H | S | — | — | — |
| 171 | | H | OEt | H | H | S | — | — | — |
| 172 | | H | Cl | H | H | S | — | — | — |
| 173 | | H | H | OMe | H | N | — | — | — |
| 174 | | H | H | OEt | H | N | — | — | — |
| 175 | | H | H | Cl | H | N | — | — | — |
| 176 | | H | H | NO₂ | H | N | — | — | — |
| 177 | | H | H | H | Me | N | — | — | — |
| 178 | | H | H | H | OMe | N | — | — | — |
| 179 | | H | H | H | Cl | N | — | — | — |
| 180 | | H | H | H | NO₂ | N | — | — | — |
| 181 | | H | Me | Cl | H | N | — | — | — |
| 182 | | H | H | Me | Cl | N | — | — | — |
| 183 | | H | Me | H | Me | N | — | — | — |
| 184 | | H | Cl | Br | H | N | — | — | — |

TABLE 7

| No. | A | R₁ | R₂ | R₃ | R₄ | Q₁ | Q₂ | Q₃ | Salt |
|---|---|---|---|---|---|---|---|---|---|
| 185 | 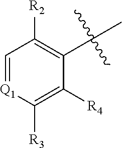 | Boc | H | H | H | N | — | — | — |
| 186 | | Boc | H | H | H | O | — | — | — |
| 187 | | Boc | H | H | H | S | — | — | — |
| 188 | | Boc | Me | H | H | N | — | — | — |
| 189 | | Boc | Et | H | H | N | — | — | — |
| 190 | | Boc | OMe | H | H | N | — | — | — |
| 191 | | Boc | OEt | H | H | N | — | — | — |
| 192 | | Boc | Cl | H | H | N | — | — | — |
| 193 | | Boc | Me | H | H | O | — | — | — |
| 194 | | Boc | Et | H | H | O | — | — | — |
| 195 | | Boc | OMe | H | H | O | — | — | — |
| 196 | | Boc | OEt | H | H | O | — | — | — |
| 197 | | Boc | Cl | H | H | O | — | — | — |
| 198 | | Boc | Me | H | H | S | — | — | — |
| 199 | | Boc | Et | H | H | S | — | — | — |
| 200 | | Boc | OMe | H | H | S | — | — | — |
| 201 | | Boc | OEt | H | H | S | — | — | — |
| 202 | | Boc | Cl | H | H | S | — | — | — |
| 203 | | Boc | H | OMe | H | N | — | — | — |
| 204 | | Boc | H | OEt | H | N | — | — | — |
| 205 | | Boc | H | Cl | H | N | — | — | — |
| 206 | | Boc | H | NO₂ | H | N | — | — | — |
| 207 | | Boc | H | H | Me | N | — | — | — |
| 208 | | Boc | H | H | OMe | N | — | — | — |
| 209 | | Boc | H | H | Cl | N | — | — | — |
| 210 | | Boc | H | H | NO₂ | N | — | — | — |
| 211 | | Boc | Me | Cl | H | N | — | — | — |
| 212 | | Boc | H | Me | Cl | N | — | — | — |
| 213 | | Boc | Me | H | Me | N | — | — | — |
| 214 | | Boc | Cl | Br | H | N | — | — | — |

The present invention relates to a compound selected from the group consisting of the following compounds, and a stereoisomer or a pharmaceutically acceptable salt thereof.
tert-butyl-4-((4-methylbenzoyl)carbamoyl)piperazine-1-carboxylate;
tert-butyl-4-((4-methoxybenzoyl)carbamoyl)piperazine-1-carboxylate;
tert-butyl-4-((4-chlorobenzoyl)carbamoyl)piperazine-1-carboxylate;
tert-butyl-4-(benzoylcarbamoyl)piperazine-1-carboxylate;
N-(4-methylbenzoyl)piperazine-1-carboxamide;
N-(4-methylbenzoyl)piperazine-1-carboxamide-2,2,2-trifluoroacetate;
N-(4-methoxybenzoyl)piperazine-1-carboxamide-2,2,2-trifluoroacetate;
N-(4-chlorobenzoyl)piperazine-1-carboxamide-2,2,2-trifluoroacetate;
N-benzoylpiperazine-1-carboxamide-2,2,2-trifluoroacetate;
N-(2-naphthoyl)piperazine-1-carboxamide-2,2,2-trifluoroacetate;
N-(4-cyanobenzoyl)piperazine-1-carboxamide-2,2,2-trifluoroacetate;
N-(furan-2-carbonyl)piperazine-1-carboxamide-2,2,2-trifluoroacetate;
N-(4-nitrobenzoyl)piperazine-1-carboxamide-2,2,2-trifluoroacetate;
N-(2-naphthoyl)piperazine-1-carboxamide hydrochloride;
N-(4-cyanobenzoyl)piperazine-1-carboxamide hydrochloride;
N-(4-nitrobenzoyl)piperazine-1-carboxamide hydrochloride;
N-benzoylpiperazine-1-carboxamide hydrochloride;
N-(furan-2-carbonyl)piperazine-1-carboxamide hydrochloride;
N-(4-methylbenzoyl)piperazine-1-carboxamide hydrochloride;
N-(4-methoxybenzoyl)piperazine-1-carboxamide hydrochloride;
N-(4-chlorobenzoyl)piperazine-1-carboxamide hydrochloride;
N-(2,4-dichlorobenzoyl)piperazine-1-carboxamide-2,2,2-trifluoroacetate;
N-(4-bromobenzoyl)piperazine-1-carboxamide-2,2,2-trifluoroacetate;
N-(thiophene-2-carbonyl)piperazine-1-carbxamide-2,2,2-trifluoroacetate;
N-(benzo[b]thiophene-2-carbonyl)piperazine-1-carboxamide-2,2,2-trifluoroacetate;
N-(4-fluorobenzoyl)piperazine-1-carboxamide-2,2,2-trifluoroacetate;
N-isonicotinoylpiperazine-1-carboxamide-2,2,2-trifluoroacetate;
N-(2,4-dichlorobenzoyl)piperazine-1-carboxamide hydrochloride;
N-(4-bromobenzoyl)piperazine-1-carboxamide hydrochloride;
N-(benzo[b]thiophene-2-carbonyl)piperazine-1-carboxamide hydrochloride;
N-isonicotinoylpiperazine-1-carboxamide hydrochloride;
N-(4-fluorobenzoyl)piperazine-1-carboxamide hydrochloride;
N-(thiophene-2-carbonyl)piperazine-1-carboxamide hydrochloride;
N-(3-chlorobenzoyl)piperazine-1-carboxamide hydrochloride;
N-(3-methylbenzoyl)piperazine-1-carboxamide hydrochloride;
N-(3-methoxybenzoyl)piperazine-1-carboxamide hydrochloride;
N-(3-chlorobenzoyl)piperazine-1-carboxamide-2,2,2-trifluoroacetate;
N-(3-methylbenzoyl)piperazine-1-carboxamide-2,2,2-trifluoroacetate; and
N-(3-methoxybenzoyl)piperazine-1-carboxamide-2,2,2-trifluoroacetate.

Further, the present invention relates to a pharmaceutical composition which includes the compound, a stereoisomer or a pharmaceutically acceptable salt thereof.

Agmatinase is an enzyme that decomposes agmatine, and it was confirmed that a content of the agmatine is reduced in some patients with autism, which is determined to be due to excessive action of the agmatinase. Further, this condition is predicted to suppress excessive excitatory nerve activity, and thus, it is expected to have preventive or therapeutic effects on emotional and behavioral disorders, as well as symptoms related thereto. Since the compound of the present invention has an agmatinase inhibitory activity, it could be understood that the above effects are expected.

The pharmaceutical composition may be a pharmaceutical composition for preventing or treating emotional and behavioral disorder-related symptoms, and specifically, the emotional and behavioral disorder-related symptoms may include lack of sociability, lack of social cognitive intelligence, stereotyped behavior, hyperactivity, impulsivity and distraction, but it is not limited thereto.

In the present invention, the lack of sociability refers to a condition in which a subject patient cannot form a smooth relationship with people normally in relationships with others who have to contact with the subject patient or are around him or her, or does not have a normal social life. For example, this disease refers to a state where a person does not fit in a relationship with people but is immersed in his or her own world.

In the present invention, the lack of social cognitive intelligence refers to a state that social cognitive intelligence, which is an inherent ability to determine lacking of social behavior helpful to adapt to interpersonal relationships or social life. For example, this refers to a state in which the behavior of understanding and inferring facial expressions, behaviors, emotions, etc. is not exhibited, and the conventions, morality and appropriate behavior in mutual relationships required by the group are not normally performed.

In the present invention, the stereotyped behavior refers to a continuous and repetitive behavior such as shaking the body back and forth, continuously moving the hands, repeating a meaningless sound, etc., a state in which the same behavior is continuously repeated regardless of the surrounding situations.

In the present invention, the hyperactivity is also referred to as excessive behavior, and refers to abnormally excessively active or moving behavior, etc., and means a state that interferes with learning and causes serious problems in behavior control.

In the present invention, the impulsivity means a tendency to act suddenly in response to internal impulses without thinking and hardly considering the consequences of actions.

In the present invention, the distraction refers to a state in which one action cannot continuously proceed, no persistence exists, and a person quickly moves to another activity or is confused about what to do.

Further, the pharmaceutical composition may be a pharmaceutical composition for preventing or treating emotional and behavioral disorders, specifically a pharmaceutical composition for preventing or treating autism spectrum disorder, schizophrenia, obsessive-compulsive disorder, depression, anxiety disorder, panic disorder and attention deficit hyperactivity disorder, but it is not limited thereto.

In the present invention, the autism type disorder is also referred to as autism spectrum disorder, which means a typical developmental disorder that exhibits serious maladaptive disorders. Fundamentally, this show deficiencies in social interaction and communication skills, limited, repetitive, and stereotyped behavioral characteristics, and very diverse characteristics in behaviors, skills, preferences, functioning, and learning that are required according to growth.

In the present invention, the schizophrenia is a disease called schizophrenia in the past, and refers to a mental disease that causes a wide range of clinical abnormalities across various aspects of personality, such as thinking, emotion, perception, behavior and the like. The schizophrenia appears in different types, and it is not a single disease but is identified as a disease group consisting of several diseases with common characteristics.

In the present invention, the obsessive-compulsive disorder refers to a subtype of anxiety disorder in which unwanted thoughts and actions are repeated, in order to reduce obsession and anxiety, which are painful thoughts, impulses, or images that repeatedly penetrate consciousness. Repetitive compulsions are the main symptom. Obsessive compulsive behaviors appear in the form of cleaning behaviors, checking behaviors, repetitive behaviors, tidying behaviors, and procrastinating behaviors. Even though they know that the behavior is undesirable and is beyond the bounds, the behavior is repeated in order to recover from anxiety due to obsessive thinking.

In the present invention, the depression does not mean a state in which only mood is temporarily lowered, but a state in which overall mental functions such as content of thought, thought process, motivation, drive, interest, behavior, sleep, and physical activity are lowered, wherein this state appears almost all day and every day.

In the present invention, the anxiety disorder refers to a mental disorder that causes impairment in daily life due to various forms of abnormal or pathological anxiety and fear, including panic disorder, agoraphobia, social anxiety disorder, specific phobia, separation anxiety disorder and the like.

In the present invention, the panic disorder is a kind of anxiety disorder, and refers to an anxiety disorder in which panic attacks, including intense anxiety that suddenly surges and intense fear that will soon die, appear repeatedly.

In the present invention, the attention deficit hyperactivity disorder, also commonly referred to as ADHD, is a mental disease in which distraction, hyperactivity, and impulsivity are seen as main symptoms, and is characterized by onset in early childhood and following a chronic course

Further, the pharmaceutical composition may be a pharmaceutical composition for preventing or treating degenerative neurological diseases.

Since the compound of the present invention can improve symptoms of sociability and social cognitive ability and has an effect of protecting nerve cells from cell damage and death caused by oxidative stress, it can exhibit medicinal effects against various degenerative neurological diseases.

The neurodegenerative diseases may include, for example, Alzheimer's disease, Parkinson's disease, multiple sclerosis, neuroblastoma, stroke, Lou Gehrig's disease, Huntington's disease, Pick's disease, Creutzfeldt-Jakob disease, post-traumatic stress disorder, depression, schizophrenia, autoimmune diseases of the nervous system, frontal temporal dementia, Lewy dementia, cortical hypoplasia, amyotrophic lateral sclerosis, and memory and cognitive decline, but they are not limited thereto.

In the present invention, the pharmaceutical composition may be prepared using a pharmaceutically suitable and physiologically acceptable additive in addition to the active ingredient, which is the compound of the present invention. The composition may be administered to a mammal. As the additive described above, for example, excipients, disintegrants, sweeteners, binders, coating agents, swelling agents, lubricants, glidants or flavoring agents may be used.

Further, the pharmaceutical composition of the present invention may be preferably formulated as a pharmaceutical composition that includes at least one pharmaceutically acceptable carrier in addition to the active ingredient in a pharmaceutically effective amount described above for administration.

The “pharmaceutically effective amount” means an amount sufficient to treat a disease with a reasonable benefit/risk ratio applicable to medical treatment, and an effective dose level may be determined based on a type and severity of patient's disease, drug activity, drug sensitivity, time of administration, route of administration and rate of excretion, duration of treatment, factors including drugs used concurrently, and other factors well known in the medical field. The pharmaceutical composition according to the present invention may be administered as an individual therapeutic agent or administered in combination with other therapeutic agents. Further, the composition may be administered sequentially or simultaneously with a conventional therapeutic agent, and may be administered in single or multiple doses. In consideration of all of the above factors, it is important to administer a minimum amount capable of attaining the maximum effect without side effects, such an amount could be easily determined by those skilled in the art.

Specifically, the effective amount of the pharmaceutical composition according to the present invention may vary depending on an age, gender, condition and/or body weight of the patient, absorption of the active ingredient in the body, inactivation rate and excretion rate, type of disease, and the drug to be used in combination. Typically, 0.001 to 150 mg, preferably 0.01 to 100 mg per 1 kg of body weight may be administered daily or every other day, or may be divided into 1 to 3 times a day. However, the dosage may be increased or decreased depending on the route of administration, severity of obesity, gender, body weight, age, etc., therefore, would not limit the scope of the present invention in any way.

Further, the “pharmaceutically acceptable” refers to a composition that is physiologically acceptable and does not usually cause allergic reactions such as gastrointestinal disorders and dizziness, or similar reactions when administered to humans.

Examples of the carrier, excipient and diluent may include lactose, dextrose, sucrose, sorbitol, mannitol, xylitol, erythritol, maltitol, starch, gum acacia, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oils. Further, fillers, anti-aggregating agents, lubricants, wetting agents, flavoring agents, emulsifying agents, and preservatives may additionally be included.

Further, the composition of the present invention may be formulated using any method known in the art in order to provide rapid, sustained or delayed release of the active ingredient after administration thereof to a subject in need of treatment using the pharmaceutical composition of the present invention including humans. The formulation may be powder, granule, tablet, emulsion, syrup, aerosol, soft or hard gelatin capsule, sterile injectable solution, or sterile powder.

The compound represented by Formula 1 according to the present invention may be prepared by any method known in various documents. In the following preparative examples, the synthetic methods for some of the compounds listed in Table 1 have been briefly described, however, they are not limited thereto.

Hereinafter, the present invention will be described in detail by means of preparative examples and examples of the present invention.

### Preparative Example

### 1. Synthesis of Compound 1

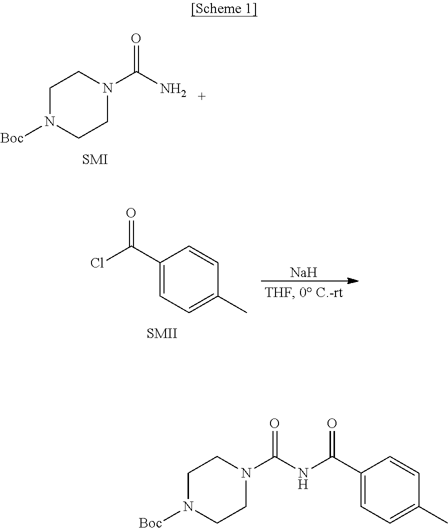

A flame-dried round bottom flask equipped with a magnetic stirrer was charged with a solution of SMI (1 mmol) and anhydrous THF (10 ml). NaH (5 mmol) was added to RM at 0° C. and stirred for about 2 h under nitrogen conditions. Then, aryl chloride (SMII, 2 mmol) in THF (5 ml) was slowly added to the reaction mixture and stirred overnight. Saturated ammonium chloride solution (7-10 ml) was added to the reaction and extracted with CH₂Cl₂. The organic layer was dried over MgSO₄, filtered and concentrated in vacuum to yield a white solid. The residue was purified using n-hexane:ethyl acetate (4:1 and 2:1) through flask column chromatography to obtain the desired product as a white solid.
Retention factor: 0.41 (H:E=1:1)
Yield: 229 mg (66%)
NMR data: 1H NMR (500 MHz, CDCl3): δ 8.44 (s, 1H), 7.76 (d, J=8.15 Hz, 2H), 7.27-7.26 (m, 2H, overlapped with CDCl3), 3.52 (s, 8H), 2.41 (s, 3H), 1.47 (s, 9H).

### 2. Synthesis of Compound 3

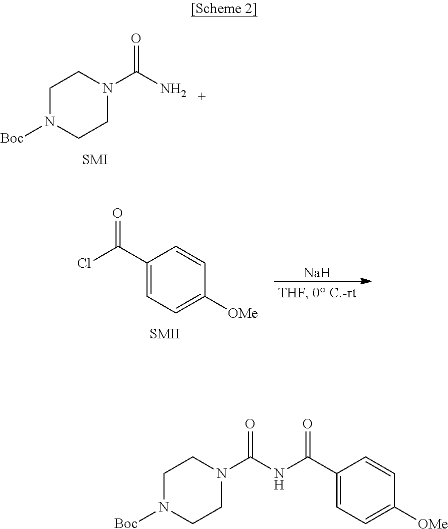

A flame dried round bottom flask equipped with a magnetic stirrer was charged with a solution of SMI (1 mmol) and anhydrous THF (10 ml). NaH (5 mmol) was added to RM at 0° C. and stirred for about 2 h under nitrogen conditions. Then, aryl chloride (SMII, 2 mmol) in THF (5 ml) was slowly added to the reaction mixture and stirred overnight. Saturated ammonium chloride solution (7-10 ml) was added to the reaction and extracted with CH₂C₂. The organic layer was dried over MgSO₄, filtered and concentrated in vacuum to yield a white solid. The residue was purified using n-hexane:ethyl acetate (4:1 and 2:1) through flask column chromatography to obtain the desired product as a white solid.
Retention factor: 0.30 (H:E=1:1)
Yield: 225 mg (62%)
NMR data: 1H NMR (500 MHz, CDCl3): δ 8.45 (s, 1H), 7.84 (d, J=8.75 Hz, 2H), 6.95 (d, J=8.8 Hz, 2H), 3.87, (s, 3H), 3.52 (s, 8H), 1.47 (s, 9H).

### 3. Synthesis of Compound 5

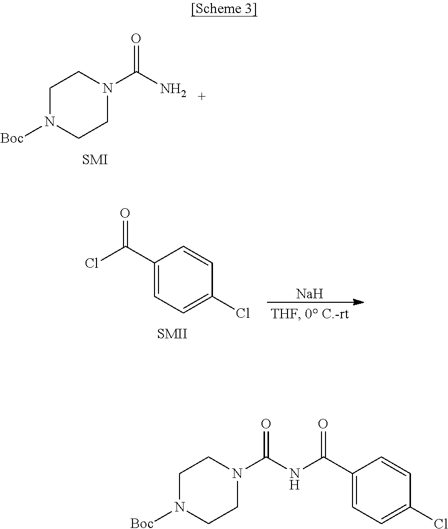

A flame dried round bottom flask equipped with a magnetic stirrer was charged with a solution of SMI (1 mmol) and anhydrous THF (10 ml). NaH (5 mmol) was added to RM at 0° C. and stirred for about 2 h under nitrogen conditions. Then, aryl chloride (SMII, 2 mmol) in THF (5 ml) was slowly added to the reaction mixture and stirred overnight. Saturated ammonium chloride solution (7-10 ml) was added to the reaction and extracted with CH₂Cl₂. The organic layer was dried over MgSO₄, filtered and concentrated in vacuum to yield a white solid. The residue was purified using n-hexane:ethyl acetate (4:1 and 2:1) through column chromatography to obtain the desired product as a white solid.
Retention factor: 0.43 (H:E=1:1)
Yield: 220 mg (60%)
NMR data: 1H NMR (500 MHz CDCl3): δ 9.13 (s, 1H), 7.85 (d, J=8.35 Hz, 2H), 7.50 (d, J=8.65 Hz, 2H), 3.52 (s, 8H), 1.48 (s, 9H).

### 4. Synthesis of Compound 6

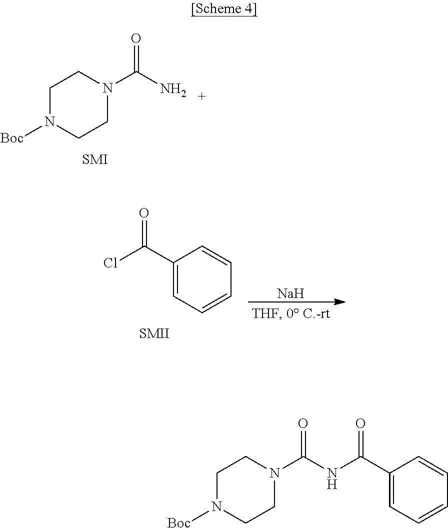

A flame dried round bottom flask equipped with a magnetic stirrer was charged with a solution of SMI (1 mmol) and anhydrous THF (10 ml). NaH (5 mmol) was added to RM at 0° C. and stirred for about 2 h under nitrogen conditions. Then, aryl chloride (SMII, 2 mmol) in THF (5 ml) was slowly added to the reaction mixture and stirred overnight. Saturated ammonium chloride solution (7-10 ml) was added to the reaction and extracted with CH₂Cl₂. The organic layer was dried over MgS₄, filtered, and concentrated in vacuum to yield a white solid. The residue was purified using n-hexane:ethyl acetate (4:1 and 2:1) through flask chromatography to obtain the desired product as a white solid.
Retention factor: 0.40 (H:E=1:1)
Yield: 234 (70%)
NMR data: 1H NMR (500 MHz, CDCl3): δ 8.32 (s, 1H), 7.85 (d, J=7.1 Hz, 2H), 7.59-7.56 (m, 1H), 7.49-7.46 (m, 2H), 3.52 (s, 8H), 1.47 (s, 9H).

### 5. Synthesis of Compound 28

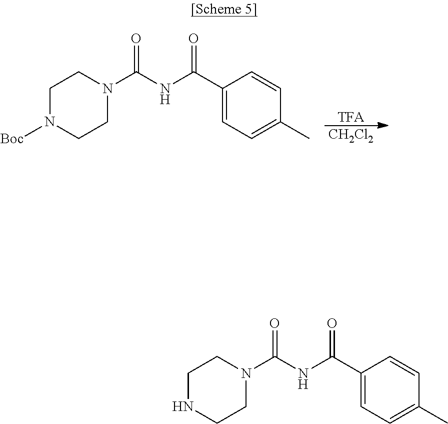

A flame dried round bottom flask equipped with a magnetic stirrer was charged with a solution of anhydrous CH₂Cl₂ (3 ml) and SMI (0.28 mmol). TFA (5.6 mmol) was added to RM and stirred overnight. Then, the RM was concentrated in vacuum and dissolved in water. The pH of the RM was made neutral using NaOH and extracted with CH₂Cl₂. The RM was dried over MgSO₄ and evaporated. Column chromatography was performed using a CH₂Cl₂:MeOH concentration gradient to obtain the desired product in a viscous liquid state.
Retention factor: 0.41 (H:E: EtOH=1:1)
Yield: 14 mg (>20%)
NMR data: 1H NMR (500 MHz, DMSO): δ 7.75-7.74 (m, 2H), 7.29-7.27 (m, 4H overlapped with CDCl3), 3.55 (s, 4H), 2.96-2.94 (m, 4H), 2.43 (s, 3H).

### 6. Synthesis of Compound 29

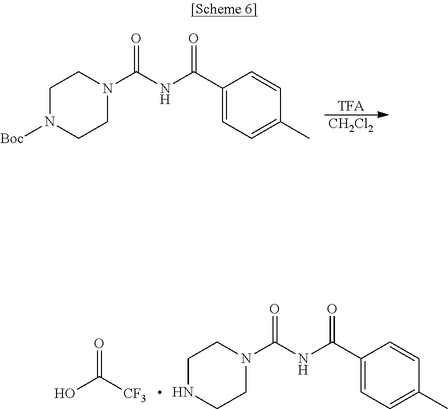

A flame dried round bottom flask equipped with a magnetic stirrer was charged with a solution of anhydrous CH₂Cl₂ (3 ml) and SMI (0.32 mmol). TFA (6.4 mmol) was added to RM and stirred overnight. Then, the RM was concentrated in vacuum using methanol and dichloromethane (DCM) to yield a solid product. The obtained product was washed with DCM and filtered to obtain the desired product as a white solid.
Retention factor: NA
Yield: 106 mg (92%)
NMR data: 1H NMR (500 MHz, DMSO): δ 10.31 (s, 1H), 9.24 (s, 2H), 7.79 (d, J=7.7 Hz, 2H), 7.30 (d, J=7.75 Hz, 2H), 3.64 (s, 4H), 3.16 (s, 4H), 2.36 (s, 3H).

### 7. Synthesis of Compound 32

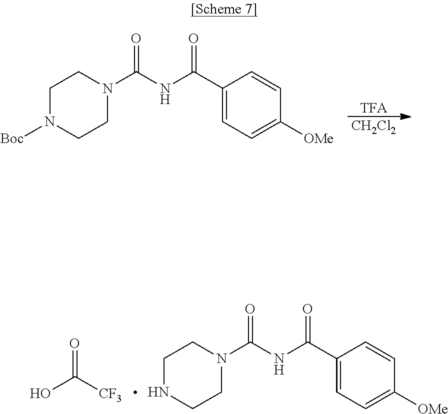

A flame dried round bottom flask equipped with a magnetic stirrer was charged with a solution of anhydrous CH₂Cl₂ (3 ml) and SMI (0.27 mmol). TFA (5.4 mmol) was added to RM and stirred overnight. Then, the RM was concentrated in vacuum using methanol and dichloromethane (DCM) to yield a solid product. The obtained product was washed with DCM and filtered to obtain the desired product as a white solid.
Retention factor: NA
Yield: 88 mg (87%)
NMR data: 1H NMR (500 MHz, DMSO): δ 10.26 (s, 1H), 9.27 (s, 2H), 7.88 (d, J=8.45 Hz, 2H), 7.01 (d, J=8.45 Hz, 2H), 3.81 (s, 3H), 3.64 (s, 4H), 3.17 (s, 4H).

### 8. Synthesis of Compound 35

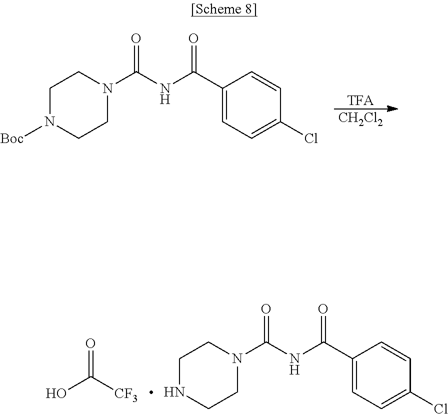

A flame dried round bottom flask equipped with a magnetic stirrer was charged with a solution of anhydrous CH₂Cl₂ (3 ml) and SMI (0.32 mmol). TFA (6.4 mmol) was added to RM and stirred overnight. Then, the RM was concentrated in vacuum using methanol and dichloromethane (DCM) to yield a solid product. The obtained product was washed with DCM and filtered to obtain the desired product as a white solid.
Retention factor: NA
Yield: 117% (96%)
NMR data: 1H NMR (500 MHz, DMSO): δ 10.48 (s, 1H), 9.16 (s, 2H), 7.88 (d, J=8.1 Hz, 2H), 7.57 (d, J=8.1 Hz, m, 2H), 3.65-3.63 (m, 4H), 3.16-3.15 (m, 4H).

### 9. Synthesis of Compound 37

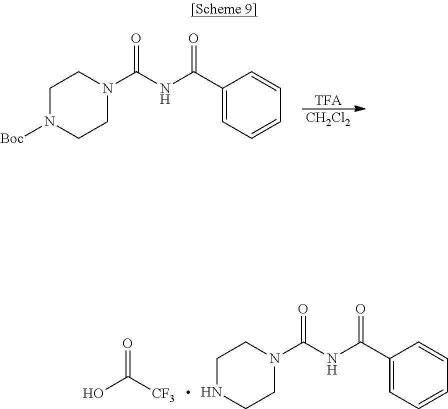

A flame dried round bottom flask equipped with a magnetic stirrer was charged with a solution of anhydrous CH₂Cl₂ (5 ml) and SMI (0.42 mmol). TFA (8.4 mmol) was added to RM and stirred overnight. Then, the RM was concentrated in vacuum using methanol and dichloromethane (DCM) to yield a solid product. The obtained product was washed with DCM and filtered to obtain the desired product as a white solid.
Retention factor: NA
Yield: 144 mg (99%)
NMR data: 1H NMR (500 MHz, DMSO): δ 10.38 (s, 1H), 9.01 (s, 2H), 7.87 (d, J=7.4 Hz, 2H), 7.62-7.59 (m, 1H), 7.52-7.49 (m, 2H), 3.65-3.63 (m, 4H), 3.17-3.15 (m, 4H).

### 10. Synthesis of Compound 76

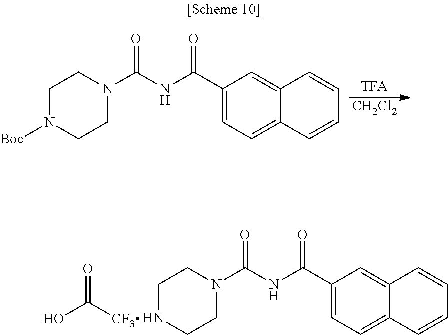

A flame dried round bottom flask equipped with a magnetic stirrer was charged with a solution of anhydrous CH₂Cl₂ (3 ml) and SMI (0.31 mmol). TFA (6.2 mmol) was added to RM and stirred overnight. Then, the RM was concentrated in vacuum using methanol and dichloromethane (DCM) to yield a solid product. The obtained product was washed with DCM and filtered to obtain the desired product as a white solid.
Retention factor: NA
Yield: 110 mg (90%)
NMR data: 1H NMR (500 MHz, DMSO): δ 10.53 (s, 1H), 8.89 (s, 2H), 8.50 (s, 1H), 8.00-7.99 (m, 3H), 7.90 (d, J=8.55 Hz, 1H), 7.66-7.60 (m, 2H), 3.67-3.65 (m, 4H), 3.17 (s, 4H).

### 11. Synthesis of Compound 39

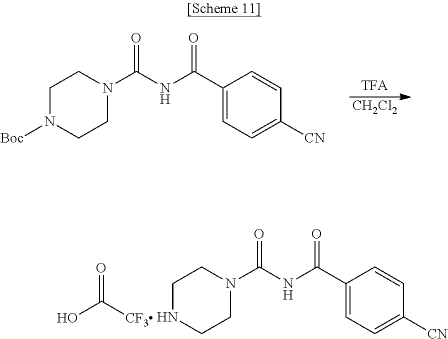

A flame dried round bottom flask equipped with a magnetic stirrer was charged with a solution of anhydrous CH₂Cl₂ (3 ml) and SMI (0.39 mmol). TFA (7.8 mmol) was added to RM and stirred overnight. Then, the RM was concentrated in vacuum using methanol and dichloromethane (DCM) to obtain a solid product. The obtained product was washed with DCM and filtered to obtain the desired product as a white solid.
Retention factor: NA
Yield: 132 mg (91%)
NMR data: 1H NMR (500 MHz, DMSO): δ 10.66 (s, 1H), 9.03 (s, 2H), 7.97 (s, 4H), 3.64-3.62 (m, 4H), 3.14 (s, 4H).

### 12. Synthesis of Compound 102

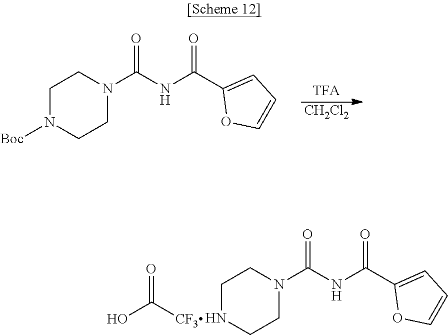

A flame dried round bottom flask equipped with a magnetic stirrer was charged with a solution of anhydrous CH₂Cl₂ (5 ml) and SMI (0.43 mmol). TFA (8.6 mmol) was added to RM and stirred overnight. Then, the RM was concentrated in vacuum using methanol and dichloromethane (DCM) to obtain a solid product. The obtained product was washed with DCM and filtered to obtain the desired product as a white solid.
Retention factor: NA
Yield: 123 mg (85%)
NMR data: 1H NMR (500 MHz, DMSO): δ 10.29 (s, 1H), 8.90 (s, 2H), 7.94 (d, J=1.65 Hz, 1H) 7.37 (d, J=3.5 Hz, 1H), 6.69-6.68 (m, 1H), 3.61-3.59 (m, 4H), 3.13 (s, 4H).

### 13. Synthesis of Compound 41

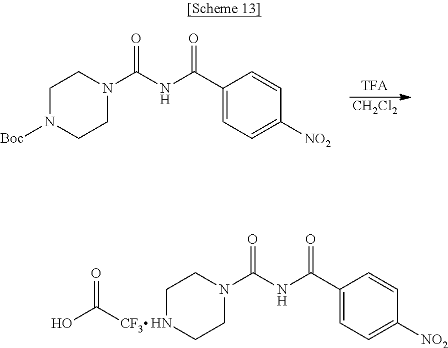

A flame dried round bottom flask equipped with a magnetic stirrer was charged with a solution of anhydrous CH₂Cl₂ (3 ml) and SMI (0.37 mmol). TFA (7.4 mmol) was added to RM and stirred overnight. Then, the RM was concentrated in vacuum using methanol and dichloromethane (DCM) to obtain a solid product. The obtained product was washed with DCM and filtered to obtain the desired product as a white solid.
Retention factor: NA
Yield: 135 mg (93%)
NMR data: 1H NMR (500 MHz, DMSO): δ 10.73 (s, 1H), 8.92 (s, 2H), 8.32 (d, J=8.55 Hz, 2H) 8.04 (d, J=8.5 Hz, 2H), 3.65-3.63 (m, 4H), 3.15 (s, 4H).

### 14. Synthesis of Compound 77

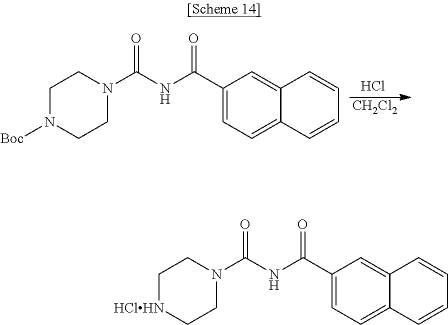

A flame dried round bottom flask equipped with a magnetic stirrer was charged with a solution of anhydrous CH₂Cl₂ (2 ml) and SMI (0.31 mmol). HCl (2 mmol) in diethyl ether (3.1 mmol) was added to RM at 0° C. and stirred overnight. Then, the RM was filtered to obtain the desired product as a white solid.
Retention factor: NA
Yield: 89 mg (90%)
NMR data: 1H NMR (500 MHz, DMSO): δ 10.56 (s, 1H), 9.47 (s, 2H), 8.54-8.53 (m, 1H), 8.05-7.99 (m, 3H), 7.93-7.91 (m, 1H), 7.67-7.60 (m, 2H), 3.72-3.70 (m, 4H), 3.14 (s, 4H); 13C NMR (125 MHz, DMSO): δ 152.6, 134.60, 131.9, 130.41, 129.07, 129.03, 128.2, 128.0, 127.6, 126.9, 124.4, 42.6.

### 15. Synthesis of Compound 40

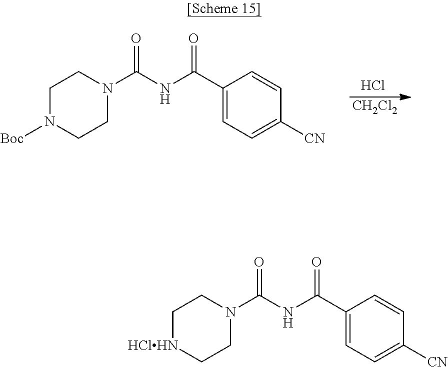

A flame dried round bottom flask equipped with a magnetic stirrer was charged with a solution of anhydrous CH₂Cl₂ (2 ml) and SMI (0.33 mmol). HCl (2 mmol) in diethyl ether (3.3 mmol) was added to RM at 0° C. and stirred overnight. Then, the RM was filtered to obtain the desired product as a white solid.
Retention factor: NA
Yield: 88.5 mg (91%)
NMR data: 1H NMR (500 MHz, DMSO): δ 10.70 (s, 1H), 9.44 (s, 2H), 8.00 (s, 4H), 3.69-3.67 (m, 4H), 3.12 (s, 4H); 13C NMR (125 MHz, DMSO): δ 166.1, 152.6, 137.8, 132.8, 129.3, 118.6, 114.8, 43.0.

### 16. Synthesis of Compound 42

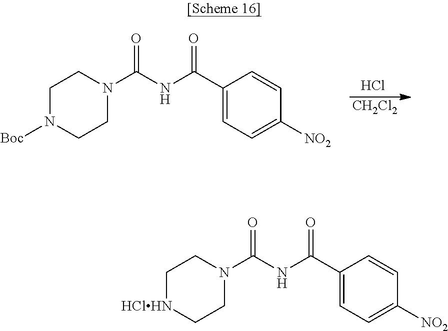

A flame dried round bottom flask equipped with a magnetic stirrer was charged with a solution of anhydrous CH₂Cl₂ (5 ml) and SMI (0.37 mmol). HCl (2 mmol) in diethyl ether (3.7 mmol) was added to RM at 0° C. and stirred overnight. Then, the RM was filtered to obtain the desired product as a white solid.
Retention factor: NA
Yield: 107 mg (92%)
NMR data: 1H NMR (500 MHz, DMSO): δ 10.79 (s, 1H), 9.48 (s, 2H), 8.32 (d, J=8.8 Hz, 2H), 8.07 (d, J=8.75 Hz, 2H), 3.71-3.69 (m, 4H), 3.13 (s, 4H); 13C NMR (125 MHz, DMSO): δ 166.0, 152.6, 149.9, 139.5, 130.0, 123.9, 43.0.

### 17. Synthesis of Compound 38

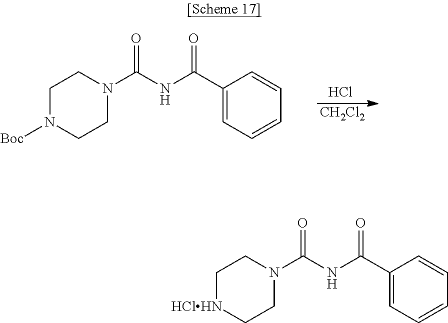

A flame dried round bottom flask equipped with a magnetic stirrer was charged with a solution of anhydrous CH₂Cl₂ (2 ml) and SMI (0.33 mmol). HCl (2 mmol) in diethyl ether (3.3 mmol) was added to RM at 0° C. and stirred overnight. Then, the RM was filtered to obtain the desired product as a white solid.
Retention factor: NA
Yield: 83 mg (93.24%)
NMR data: 1H NMR (500 MHz, DMSO): δ 10.42 (s, 1H), 9.57 (s, 2H), 7.88-7.87 (m, 2H), 7.61-7.58 (m, 1H), 7.51-7.48 (m, 2H), 3.69-3.67 (m, 4H), 3.13-3.11 (m, 4H).

### 18. Synthesis of Compound 103

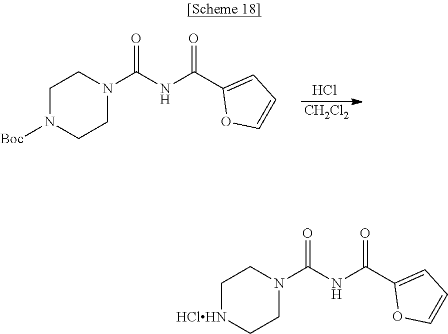

A flame dried round bottom flask equipped with a magnetic stirrer was charged with a solution of anhydrous CH₂Cl 2 (2 ml) and SMI (0.40 mmol). HCl (2 mmol) in diethyl ether (4 mmol) was added to RM at 0° C. and stirred overnight. Then, the RM was filtered to obtain the desired product as a white solid.
Retention factor: NA
Yield: 91.5 mg (88%)
NMR data: 1H NMR (500 MHz, DMSO): δ 10.32 (s, 1H), 9.49 (s, 2H), 7.93 (d, J=1.5 Hz, 1H), 7.42 (d, J=3.4 Hz, 1H), 6.67 (dd, J=3.55, 1.7 Hz, 1H), 3.66-3.64 (m, 4H), 3.10-3.09 (m, 4H); 13C NMR (125 MHz, DMSO): δ 156.6, 151.9, 146.7, 146.1, 116.5, 112.2, 42.5.

### 19. Synthesis of Compound 30

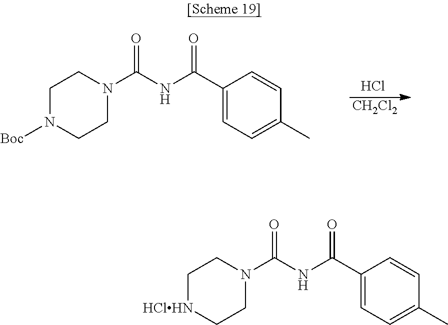

A flame dried round bottom flask equipped with a magnetic stirrer was charged with a solution of anhydrous CH₂Cl₂ (2 ml) and SMI (0.28 mmol). HCl (2 mmol) in diethyl ether (2.8 mmol) was added to RM at 0° C. and stirred overnight. Then, the RM was filtered to obtain the desired product as a white solid.
Retention factor: NA
Yield: 73 mg (92%)
NMR data: 1H NMR (500 MHz, DMSO): δ 10.30 (s, 1H), 9.44 (s, 2H), 7.78 (d, J=8.2 Hz, 2H), 7.30-7.29 (m, 2H), 3.67-3.66 (m, 4H), 3.11 (s, 4H), 2.36 (s, 3H); 13C NMR (125 MHz, DMSO): δ 166.3, 152.7, 142.6, 130.2, 128.9, 128.2, 42.5, 21.0.

### 20. Synthesis of Compound 33

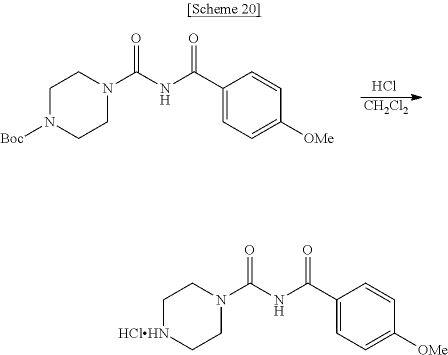

A flame dried round bottom flask equipped with a magnetic stirrer was charged with a solution of anhydrous CH₂Cl₂ (2 ml) and SMI (0.31 mmol). HCl (2 mmol) in diethyl ether (3.1 mmol) was added to RM at 0° C. and stirred overnight. Then, the RM was filtered to obtain the desired product as a white solid.
Retention factor: NA
Yield: 84.56 mg (91%)
NMR data: 1H NMR (500 MHz, DMSO): δ 10.22 (s, 1H), 9.45 (s, 2H), 7.88 (d, J=8.85 Hz, 2H), 7.02 (d, J=8.9 Hz, 2H), 3.82 (s, 3H), 3.66-3.64 (m, 4H), 3.11 (s, 4H); 13C NMR (125 MHz, DMSO): δ 166.7, 162.5, 152.9, 130.3, 125.0, 113.6, 55.5, 42.5.

### 21. Synthesis of Compound 36

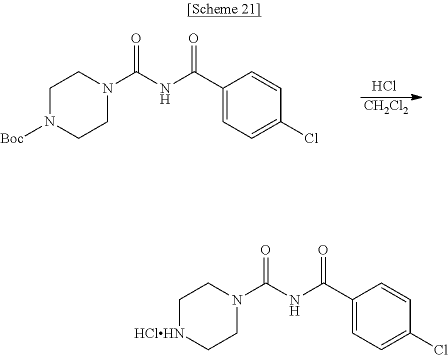

A flame dried round bottom flask equipped with a magnetic stirrer was charged with a solution of anhydrous CH₂Cl₂ (2 ml) and SMI (0.38 mmol). HCl (2 mmol) diethyl ether (3.8 mmol) was added to RM at 0° C. and stirred overnight. Then, the RM was filtered to obtain the desired product as a white solid.
Retention factor: NA
Yield: 106 mg (92%)
NMR data: 1H NMR (500 MHz, DMSO): δ 10.50 (s, 1H), 9.50 (s, 2H), 7.89 (d, J=8.86 Hz, 2H), 7.57 (d, J=8.6 Hz, 2H), 3.68-3.66 (m, 4H), 3.11 (s, 4H); 13C NMR (125 MHz, DMSO): δ 165.6, 152.4, 137.1, 131.9, 130.1, 128.4, 42.5.

### 22. Synthesis of Compound 43

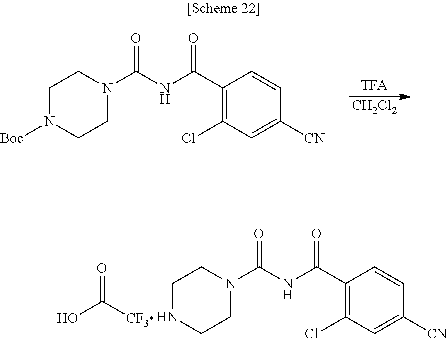

A flame dried round bottom flask equipped with a magnetic stirrer was charged with a solution of anhydrous CH₂Cl₂ (5 ml) and SMI (0.38 mmol). TFA (7.6 mmol) was added to RM and stirred overnight. Then, the RM was concentrated in vacuum using methanol and dichloromethane (DCM) to yield a solid product. The obtained product was washed with DCM and filtered to obtain the desired product as a white solid.
Retention factor: NA
Yield: 142 mg (90%)
NMR data: 1H NMR (500 MHz, DMSO): δ 10.73 (s, 1H), 9.04 (s, 2H), 7.63 (s, 1H), 7.48-7.43 (m, 2H), 3.58-3.56 (m, 4H), 3.08 (s, 4H).

### 23. Synthesis of Compound 45

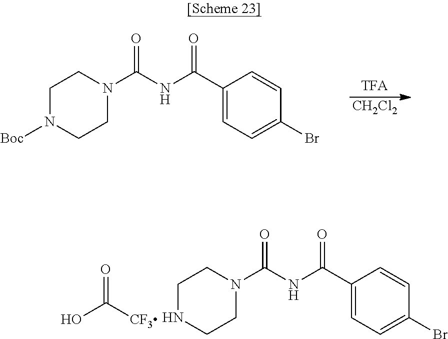

A flame dried round bottom flask equipped with a magnetic stirrer was charged with a solution of anhydrous CH₂Cl₂ (5 ml) and SMI (0.26 mmol). TFA (5.2 mmol) was added to RM and stirred overnight. Then, the RM was concentrated in vacuum using methanol and dichloromethane (DCM) to yield a solid product. The obtained product was washed with DCM and filtered to obtain the desired product as a white solid.
Retention factor: NA
Yield: 97.5 mg (88%)
NMR data: 1H NMR (500 MHz, DMSO): δ 10.48 (s, 1H), 9.14 (s, 2H), 7.80-7.71 (m, 4H), 3.64 (s, 4H), 3.16 (s, 4H).

### 24. Synthesis of Compound 104

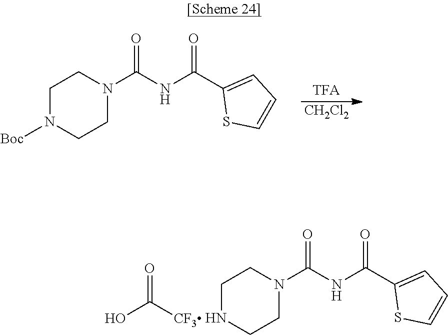

A flame dried round bottom flask equipped with a magnetic stirrer was charged with a solution of anhydrous CH₂Cl₂ (5 ml) and SMI (0.32 mmol). TFA (6.4 mmol) was added to RM and stirred overnight. Then, the RM was concentrated in vacuum using methanol and dichloromethane (DCM) to yield a solid product. The obtained product was washed with DCM and filtered to obtain the desired product as a white solid.
Retention factor: NA
Yield: 104 mg (92%)
NMR data: 1H NMR (500 MHz, DMSO): δ 10.42 (s, 1H), 9.04 (s, 2H), 7.94-7.86 (m, 2H), 7.19-7.16 (m, 1H), 3.64-3.62 (m, 4H), 3.16-3.14 (m, 4H).

### 25. Synthesis of Compound 129

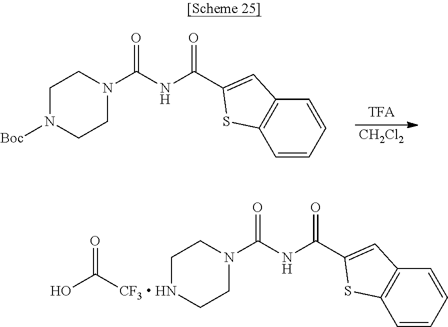

A flame dried round bottom flask equipped with a magnetic stirrer was charged with a solution of anhydrous CH₂Cl₂ (5 ml) and SMI (0.23 mmol). TFA (4.6 mmol) was added to RM and stirred overnight. Then, the RM was concentrated in vacuum using methanol and dichloromethane (DCM) to yield a solid product. The obtained product was washed with DCM and filtered to obtain the desired product as a white solid.
Retention factor: NA
Yield: 79 mg (85%)
NMR data: 1H NMR (500 MHz, DMSO): δ 10.68 (s, 1H), 8.97 (s, 2H), 8.31 (s, 1H), 8.07-8.05 (m, 1H), 7.08-7.98 (m, 1H), 7.53-7.42 (m, 2H), 3.67-3.65 (m, 4H), 3.17 (s, 4H).

### 26. Synthesis of Compound 47

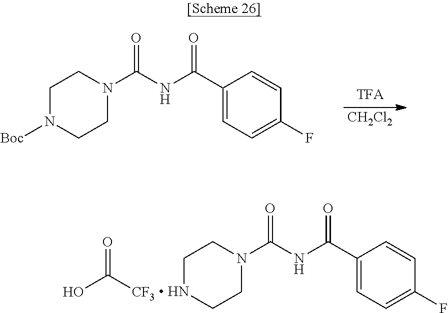

A flame dried round bottom flask equipped with a magnetic stirrer was charged with a solution of anhydrous CH₂Cl₂ (5 ml) and SMI (0.36 mmol). TFA (7.2 mmol) was added to RM and stirred overnight. Then, the RM was concentrated in vacuum using methanol and dichloromethane (DCM) to yield a solid product. The obtained product was washed with DCM and filtered to obtain the desired product as a white solid.
Retention factor: NA
Yield: 105 mg (80%)
NMR data: 1H NMR (500 MHz, DMSO): δ 10.42 (s, 1H), 9.08 (s, 2H), 7.96-7.94 (m, 2H), 7.35-7.27 (m, 2H), 3.65-3.63 (m, 4H), 3.16 (s, 4H).

### 27. Synthesis of Compound 154

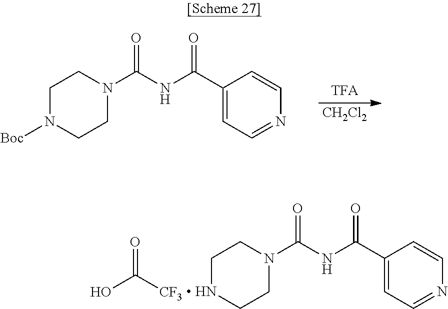

A flame dried round bottom flask equipped with a magnetic stirrer was charged with a solution of anhydrous CH₂Cl₂ (5 ml) and SMI (0.50 mmol). TFA (10 mmol) was added to RM and stirred about overnight. Then, the RM was concentrated in vacuum using methanol and dichloromethane (DCM) to yield a solid product. The obtained product was washed with DCM and filtered to obtain the desired product as a white solid.
Retention factor: NA
Yield: 150 mg (86.13)
NMR data: 1H NMR (500 MHz, DMSO): δ 10.82 (s, 1H), 9.31 (s, 2H), 8.80 (s, 2H), 7.82 (s, 2H), 3.67 (s, 4H), 3.17 (s, 4H).

### 28. Synthesis of Compound 44

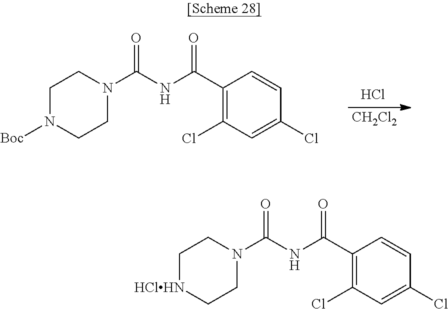

A flame dried round bottom flask equipped with a magnetic stirrer was charged with a solution of anhydrous CH₂Cl₂ (2 ml) and SMI (0.20 mmol). HCl (2 mmol) in diethyl ether (1 ml) was added to RM at 0° C. and stirred overnight. Then, the RM was filtered to obtain the desired product as a white solid.
Retention factor: NA
Yield: 62 mg (91.55%)
NMR data: 1H NMR (500 MHz, DMSO): δ 10.79 (s, 1H), 9.46 (s, 2H), 7.69 (d, J=1.85 Hz, 1H), 7.54-7.48 (m, 2H), 3.66-3.64 (m, 4H), 3.10-3.08 (m, 4H).

### 29. Synthesis of Compound 46

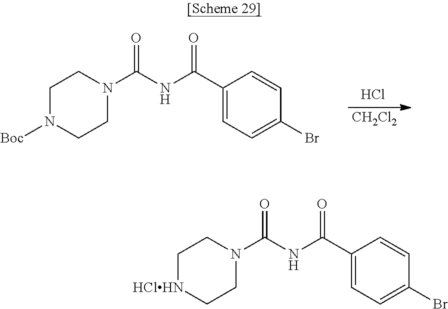

A flame dried round bottom flask equipped with a magnetic stirrer was charged with a solution of anhydrous CH₂Cl₂ (2 ml) and SMI (0.19 mmol). HCl (2 mmol) in diethyl ether (0.95 ml) was added to RM at 0° C. and stirred overnight. Then, the RM was filtered to obtain the desired product as a white solid.
Retention factor: NA
Yield: 61 mg (92.10%)
NMR data: 1H NMR (500 MHz, DMSO): δ 10.51 (s, 1H), 9.53 (s, 2H), 7.81 (d, J=8.6 Hz, 2H), 7.70 (d, J=8.6 Hz, 2H), 3.68-3.66 (m, 4H), 3.10 (s, 4H); 13C NMR (125 MHz, DMSO): δ 165.8, 152.4, 132.3, 131.4, 130.2, 126.2, 42.5.

30. Synthesis of Compound 130

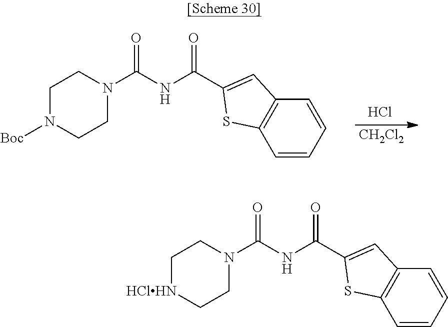

A flame dried round bottom flask equipped with a magnetic stirrer was charged with a solution of anhydrous CH₂Cl₂ (2 ml) and SMI (0.21 mmol). HCl (2 mmol) in diethyl ether (1.05 ml) was added to RM at 0° C. and stirred overnight. Then, the RM was filtered to obtain the desired product as a white solid.
Retention factor: NA
Yield: 60 mg (87.70%)
NMR data: 1H NMR (500 MHz, DMSO): δ 10.76 (s, 1H), 9.53 (s, 2H), 8.38 (s, 1H), 8.06-8.04 (m, 1H), 8.00-7.97 (m, 1H), 7.52-7.45 (m, 2H), 3.72-3.69 (m, 4H), 3.15-3.13 (m, 4H).

### 31. Synthesis of Compound 155

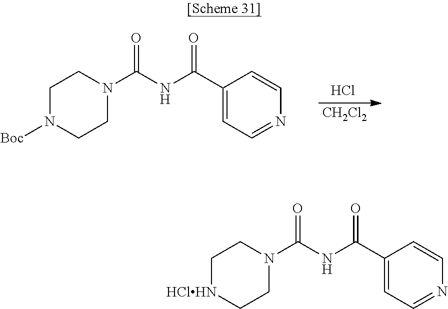

A flame dried round bottom flask equipped with a magnetic stirrer was charged with a solution of anhydrous CH₂Cl₂ (2 ml) and SMI (1 mmol). HCl (2 mmol) in diethyl ether (2 ml) was added to RM at 0° C. and stirred overnight. Then, the RM was filtered to obtain the desired product as a white solid.
Retention factor: NA
Yield: 97 mg (89.50%)
NMR data: 1H NMR (500 MHz, DMSO): δ 11.17 (s, 1H), 9.40 (s, 2H), 8.98-8.95 (m, 2H), 8.16-8.14 (m, 2H), 3.53-3.51 (m, 4H), 3.02-2.98 (m, 4H).

### 32. Synthesis of Compound 48

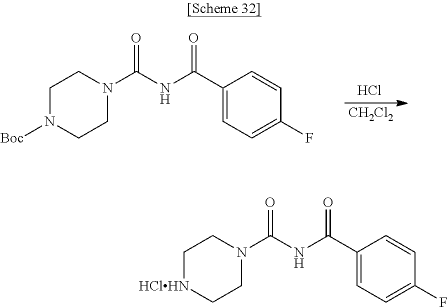

A flame-dried round bottom flask equipped with a magnetic stirrer was charged with a solution of anhydrous CH₂Cl₂ (2 ml) and SMI (0.31 mmol), HCl (2 mmol) in diethyl ether (3.1 mmol) was added to RM at 0° C. and stirred overnight at room temperature. Then, the RM was filtered to obtain the desired product as a white solid.
Retention factor: NA
Yield: >90%
NMR data: 1H NMR (500 MHz, DMSO): δ 10.45 (s, 1H), 9.51 (m, 2H), 7.97-7.95 (m, 2H), 7.35-7.32 (m, 2H), 3.69-3.66 (m, 4H), 3.12-3.10 (m, 4H).

### 33. Synthesis of Compound 105

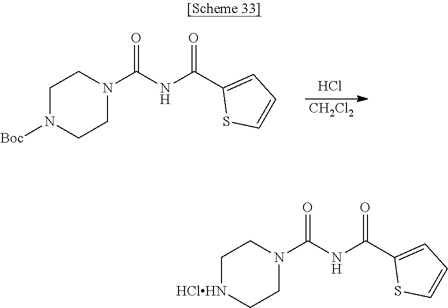

A flame-dried round bottom flask equipped with a magnetic stirrer was charged with a solution of anhydrous CH₂Cl₂ (2 ml) and SMI (0.35 mmol), and HCl (2 mmol) in diethyl ether (3.5 mmol) was added to RM at 0° C. and stirred overnight at room temperature. Then, the RM was filtered to obtain the desired product as a white solid.
Retention factor: NA
Yield: >90%
NMR data: 1H NMR (500 MHz, DMSO): δ 10.46 (s, 1H), 9.35 (s, 2H), 8.02-8.01 (m, 1H), 7.92-7.91 (m, 1H), 7.21 (dd, J=5, 3.8 Hz, 1H), 3.69-3.66 (m, 4H), 3.12-3.10 (m, 4H).

### 34. Synthesis of Compound 51

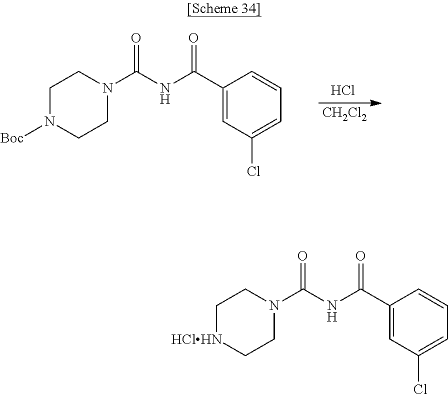

A flame-dried round bottom flask equipped with a magnetic stirrer was charged with a solution of anhydrous CH₂Cl₂ (2 ml) and SMI (0.30 mmol), HCl (2 mmol) in diethyl ether (3.0 mmol) was added to RM at 0° C. and stirred overnight at room temperature. Then, the RM was filtered to obtain the desired product as a white solid.
Retention factor: NA
Yield: >90%
NMR data: 1H NMR (500 MHz, DMSO): δ 10.54 (s, 1H), 9.52 (s, 2H), 7.92-7.91 (m, 1H), 7.84-7.82 (m, 1H), 7.68-7.65 (m, 1H) 7.55-7.52 (m, 1H), 3.69-3.67 (m, 4H), 3.10 (s, 4H).

### 35. Synthesis of Compound 53

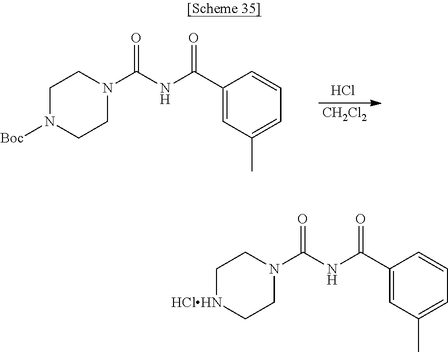

A flame-dried round bottom flask equipped with a magnetic stirrer was charged with a solution of anhydrous CH₂Cl₂ (2 ml) and SMI (0.30 mmol), HCl (2 mmol) in diethyl ether (3.0 mmol) was added to RM at 0° C. and stirred overnight at room temperature. Then, the RM was filtered to obtain the desired product as a white solid.
Retention factor: NA
Yield: >85%
NMR data: 1H NMR (500 MHz, DMSO): δ 10.39 (s, 1H), 9.66 (s, 2H), 7.74-7.69 (m, 2H), 7.43-7.37 (m, 2H), 3.71-3.69 (m, 4H), 3.17-3.13 (m, 4H), 2.37 (s, 3H).

### 36. Synthesis of Compound 55

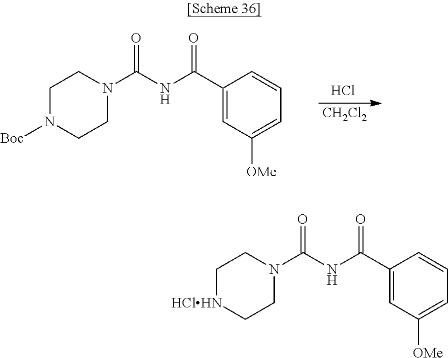

A flame-dried round bottom flask equipped with a magnetic stirrer was charged with a solution of anhydrous CH₂Cl₂ (2 ml) and SMI (0.37 mmol), and HCl (2 mmol) in diethyl ether (3.7 mmol) was added to RM at 0° C. and stirred overnight at room temperature. Then, the RM was filtered to obtain the desired product as a white solid.
Retention factor: NA
Yield: >85%
NMR data: 1H NMR (500 MHz, DMSO): δ 10.43 (s, 1H), 9.65 (s, 2H), 7.48-7.39 (m, 3H), 7.17-7.15 (m, 1H), 3.80 (s, 3H), 3.69-3.67 (m, 4H), 3.12-3.09 (m, 4H).

### 37. Synthesis of Compound 52

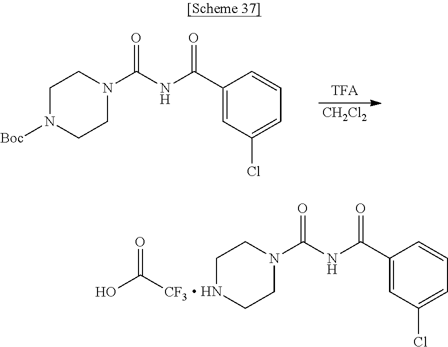

A flame dried round bottom flask equipped with a magnetic stirrer was charged with a solution of anhydrous CH₂Cl₂ (2 ml) and SMI (0.43 mmol). TFA (8.6 mmol) was added to RM at 0° C. and stirred overnight at room temperature. Then, the RM was concentrated in vacuum using methanol and dichloromethane (DCM) to obtain a solid product. The obtained product was washed with DCM and filtered to obtain the desired product as a white solid.
Retention factor: NA
Yield: >90%
NMR data: 1H NMR (500 MHz, DMSO): δ 10.52 (s, 1H), 9.19 (s, 2H), 7.90-7.89 (m, 1H), 7.83-7.81 (m, 1H), 7.68-7.66 (m, 1H) 7.56-7.52 (m, 1H), 3.66-3.64 (m, 4H), 3.17-3.16 (m, 4H).

### 38. Synthesis of Compound 54

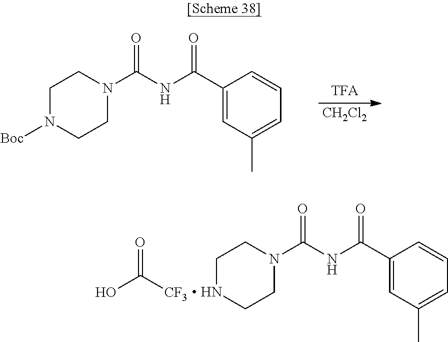

A flame dried round bottom flask equipped with a magnetic stirrer was charged with a solution of anhydrous CH₂Cl₂ (2 ml) and SMI (0.40 mmol). TFA (8 mmol) was added to RM at 0° C. and stirred overnight at room temperature. Then, the RM was concentrated in vacuum using methanol and dichloromethane (DCM) to obtain a solid product. The obtained product was washed with DCM and filtered to obtain the desired product as a white solid.
Retention factor: NA
Yield: >85%
NMR data: 1H NMR (500 MHz, DMSO): δ 10.37 (s, 1H), 9.31 (s, 2H), 7.70-7.65 (m, 2H), 7.40-7.35 (m, 2H), 3.66-3.64 (m, 4H), 3.16-3.14 (m, 4H), 2.35 (s, 3H).

### 39. Synthesis of Compound 56

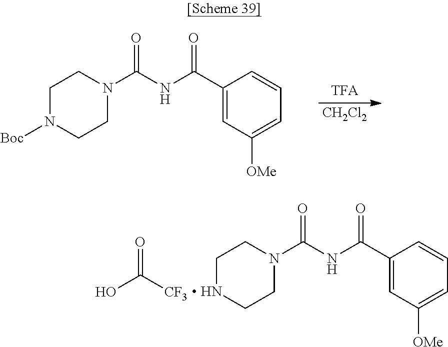

A flame dried round bottom flask equipped with a magnetic stirrer was charged with a solution of anhydrous CH₂Cl₂ (2 ml) and SMI (0.39 mmol). TFA (7.8 mmol) was added to RM at 0° C. and stirred overnight at room temperature. Then, the RM was concentrated in vacuum using methanol and dichloromethane (DCM) to obtain a solid product. The obtained product was washed with DCM and filtered to obtain the desired product as a white solid.
Retention factor: NA
Yield: >85%
NMR data: 1H NMR (500 MHz, DMSO): δ 10.39 (s, 1H), 9.23 (s, 2H), 7.47-7.39 (m, 3H), 7.17-7.15 (m, 1H), 3.80 (s, 3H), 3.65-3.63 (m, 4H), 3.16-3.14 (m, 4H).

### Example

### 1. Confirmation of Agmatinase Inhibitory Activity of the Compound—Using Urea Assay

### (1) Experimental Reagents and Manufacturing Methods

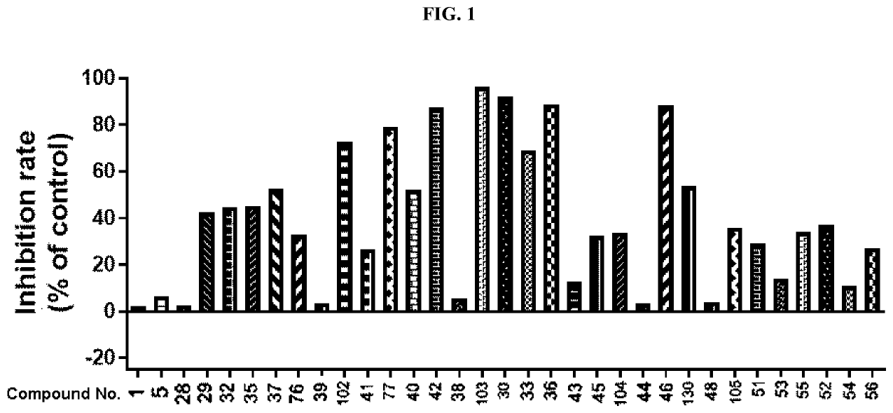

The agmatinase inhibitory activity (FIG. 1) was measured for some of the compounds of the present invention shown in Table 1.

Mg containing buffer was used as the assay buffer, and agmatinase (speB) and recombinant protein were used at 0.1 ml (final concentration: 2 mg/ml

### (2) Experimental Method and Result

After adding the sample and agmatinase to the activity buffer and performing a reaction, 1 ul (final conc. 500 uM) of agmatine was added. Thereafter, the assay was performed to measure the absorbance, and the amount was converted according to the standard curve.

Calculation of the urea concentration was calculated using Equation 1 below.

$$[ Urea ] = \frac{{OD}_{SAMPLE} - {OD}_{BLANK}}{{OD}_{STANDARD} - {OD}_{BLANK}} \times n \times [ STD ] ( mg / dL ) \qquad [ Equation 1 ]$$

After treatment at concentrations of 1 uM, 10 uM, 30 uM, 50 uM and 300 uM for each compound, the agmatinase inhibitory activity was determined in percent. As shown in FIG. 1, it can be seen that the higher the concentration, the larger the agmatinase inhibitory activity.

As shown in FIG. 1, it can be seen that most of the compounds of the present invention exhibited agmatinase inhibitory activity.

### 2. Confirmation of Sociability and Cognitive Improvement Effects of Compounds in Autism Animal Models

In order to confirm therapeutic effects of the compound according to the present invention on lack of sociability, which is one of the phenotypes of developmental disorders, a three-chamber test was performed using a valproic acid (VPA)-induced autism animal model. The chamber duration is shown as stranger (retention time to mouse), empty (retention time to empty space), and center (retention time to center space), and if the mouse stay time is longer than empty space, it is determined to be social.

As a result of the experiment, in the case of VPA mice, the retention time to empty space was longer than the retention time to mouse, which means that sociality was reduced.

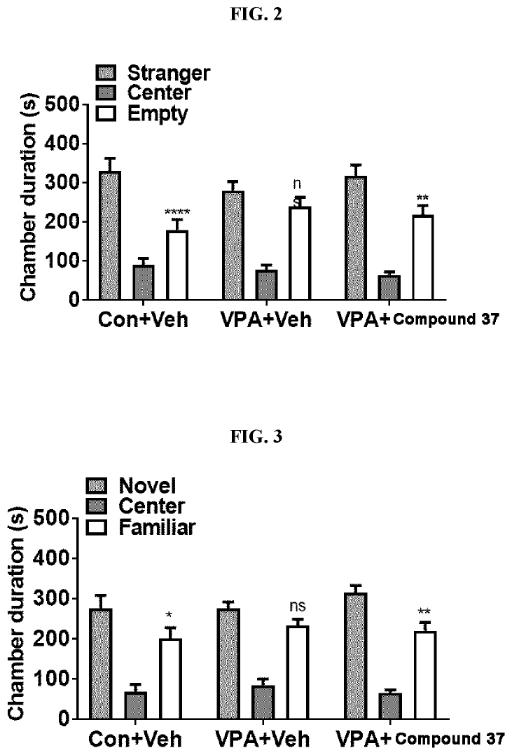

On the other hand, when VPA mice were treated with the compound of the present invention, it was confirmed that the retention time to mouse was higher than the retention time to empty space, which means that the sociality of the VPA mice was improved by the compound treatment (FIG. 2).

A stimulus mouse (familiar) was explored for 10 minutes in the experimental animals, a new stimulus mouse (novel) was added to the experimental animals. Then, after 10 minutes of exploration again, the result of evaluating whether or not the novel mouse spent more time than the familiar mouse was confirmed (FIG. 3). At this time, the old mouse is called familiar, and the newly introduced mouse is called novel (if a spending time with the novel mouse is longer than that of the familiar mouse, it is determined that there is a social cognitive ability). In other words, it is determined that it has the cognitive ability to distinguish between the existing familiar mouse and the new novel mouse.

The experimental results were expressed by measuring a time for the test mice to explore each mouse or a sniffing time for each mouse of the test mice. It means that the more time spent with the novel mouse than the familiar mouse or the more time spent sniffing the novel mouse than the familiar mouse, the higher the social preference.

As a result of the experiment, in the case of VPA mice, they spent more time with familiar mice than with novel mice, and spent more time sniffing with familiar mice than with novel mice, which means that their social preference was reduced.

On the other hand, when VPA mice were treated with the compound of the present invention, more time was spent with novel mice than familiar mice, and sniffing time was increased for novel mice than for familiar mice. Through this, it can be seen that treatment with the compound of the present invention can improve social preference (FIGS. 2 to 3).

Through the results of confirming sociability and social preference, when sociability and social preference are improved, symptoms of sociability and social cognitive intelligence are improved, and when these symptoms are improved, there will be effects of preventing or treating not only emotional and behavioral disorders such as autism spectrum disorder and schizophrenia, but also cognitive related intellectual disability or degenerative neurological diseases such as Alzheimer's disease and Parkinson's diseases, such that it may be predicted that administering the compound of the present invention to a patient group with emotional and behavioral disorder can achieve preventive or therapeutic effects.

### Repetitive Behavior Experiment

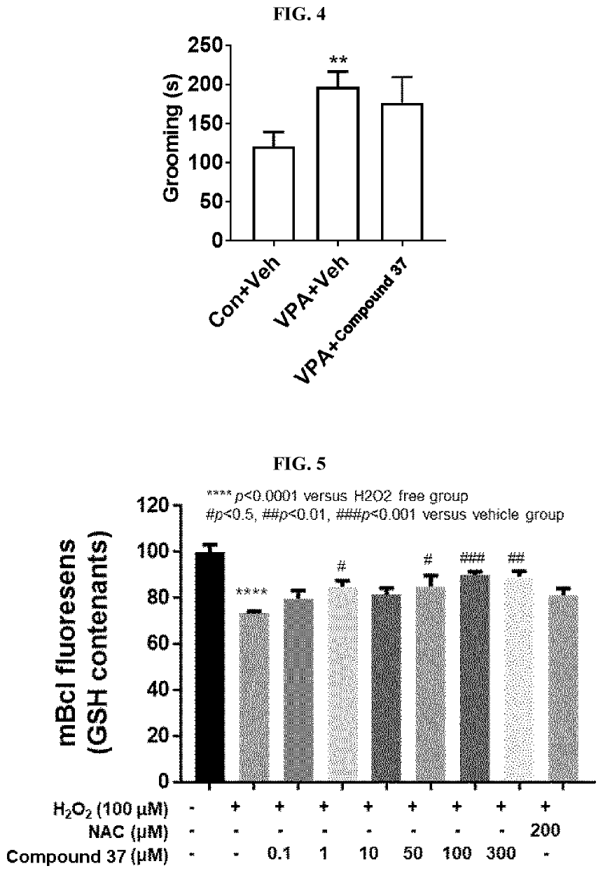

In order to confirm the therapeutic effect of the compound according to the present invention on homology, one of the phenotypes of developmental disorders, a self-grooming test was performed using a valproic acid (VPA)-induced autism animal model (FIG. 4).

The animals were placed in an empty cage, and the duration of licking or grooming (specified as self-grooming) per hour was measured and graphed. If the duration is increased, it means that repetitive behavior appears.

Through the results of demonstrating repetitive behavior, if the repetitive behavior is improved, the symptoms of stereotyped behavior and impulsivity may be improved. When the symptoms of stereotyped behavior are improved, the effect of preventing or treating emotional and behavioral disorders such as autism spectrum disorder and obsessive-compulsive disorder may be expected. Further, if the symptoms of impulsivity are improved, there will be a preventive or therapeutic effect on emotional and behavioral disorders such as autism spectrum disorder, obsessive-compulsive disorder, depression, anxiety disorder, panic disorder, and attention deficit hyperactivity disorder. Therefore, it can be predicted that administering the compound to a patient group with the emotional and behavioral disorder will attain preventive or therapeutic effects.

### GSH Assay

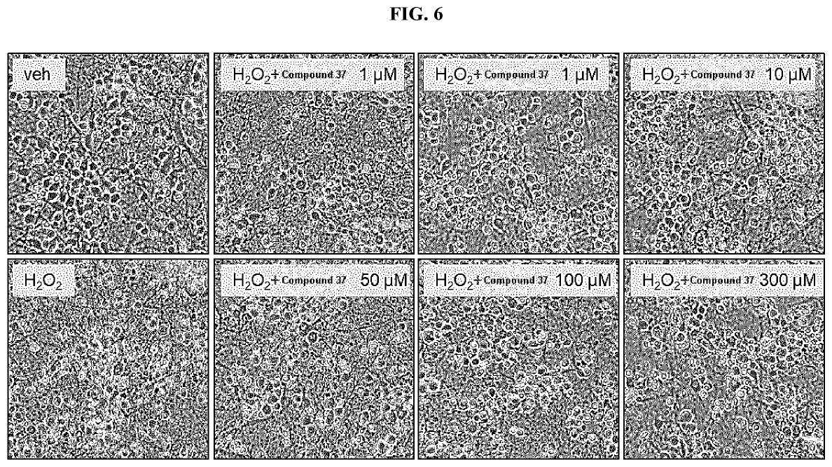

In order to confirm the neuroprotective effect of the compound according to the present invention, after 24 hours of pretreatment, cell oxidation stimulation was induced with H₂O₂, and after performing the GSH assay (FIG. 5), cell images were confirmed (FIG. 6). The cells used herein were primary nerve cells, and were treated with 0.1, 1, 10, 50, 100, and 300 μM of the compound of the present invention.

1: A compound represented by Formula 1 below, a stereoisomer or a pharmaceutically acceptable salt thereof: 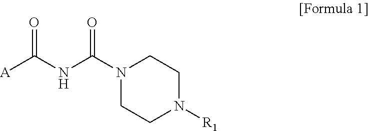
wherein R₁ is hydrogen or a tert-butyloxycarbonyl group,
A is a single or double cyclic group of C₅-C₁₀,
each ring of the cyclic group is unsubstituted or substituted with 1 to 3 heteroatoms, and
the cyclic group is unsubstituted or substituted with halogen, C₁-C₅ alkyl, C₁-C₅ alkoxy, CN or NO₂.

2: The compound, a stereoisomer or a pharmaceutically acceptable salt thereof according to claim 1, wherein A is selected from the group consisting of the following cyclic groups: 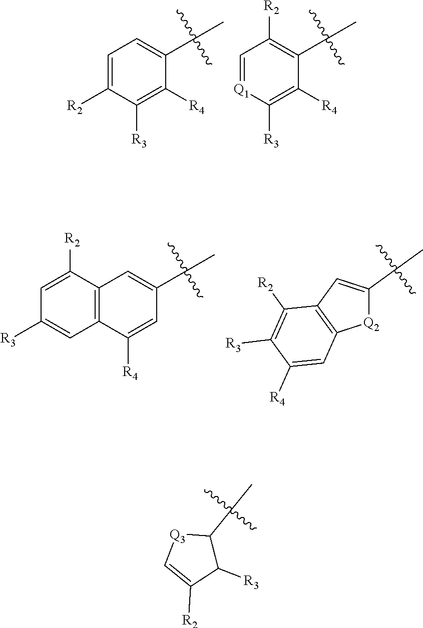
wherein R₂, R₃ and R₄ are each independently hydrogen, halogen, C₁-C₅ alkyl, C₁-C₅ alkoxy, CN or NO₂, and
Q₁, Q₂ and Q₃ are each independently N, O or S.

3: The compound, a stereoisomer or a pharmaceutically acceptable salt thereof according to claim 1, wherein A is selected from the group consisting of the following cyclic groups: 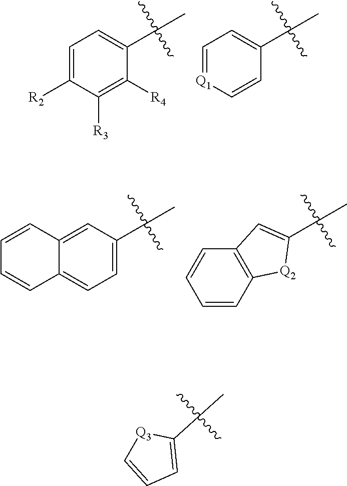
wherein R₂, R₃ and R₄ are each independently hydrogen, halogen, C₁-C₅ alkyl, C₁-C₅ alkoxy, CN or NO₂; and
Q₁, Q₂ and Q₃ are each independently N, O or S.

4: The compound, a stereoisomer or a pharmaceutically acceptable salt thereof according to claim 1, wherein A is selected from the group consisting of the following cyclic groups: 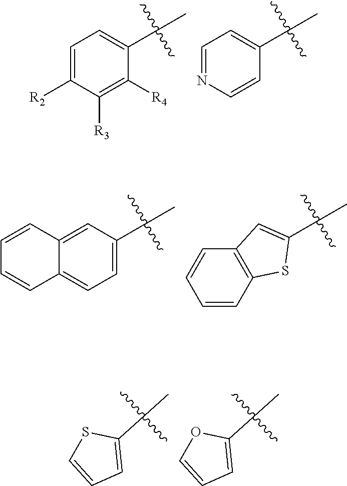
wherein R₂, R₃ and R₄ are each independently hydrogen, halogen, C₁-C₅ alkyl, C₁-C₅ alkoxy, CN or NO₂.

5: The compound, a stereoisomer or a pharmaceutically acceptable salt thereof according to claim 1, wherein the compound is selected from the group consisting of the following compounds:
tert-butyl-4-((4-methylbenzoyl)carbamoyl)piperazine-1-carboxylate;
tert-butyl-4-((4-methoxybenzoyl)carbamoyl)piperazine-1-carboxylate;
tert-butyl-4-((4-chlorobenzoyl)carbamoyl)piperazine-1-carboxylate;
tert-butyl-4-(benzoylcarbamoyl)piperazine-1-carboxylate;
N-(4-methylbenzoyl)piperazine-1-carboxamide;
N-(4-methylbenzoyl)piperazine-1-carboxamide-2,2,2-trifluoroacetate;
N-(4-methoxybenzoyl)piperazine-1-carboxamide-2,2,2-trifluoroacetate;
N-(4-chlorobenzoyl)piperazine-1-carboxamide-2,2,2-trifluoroacetate;
N-benzoylpiperazine-1-carboxamide-2,2,2-trifluoroacetate;
N-(2-naphthoyl)piperazine-1-carboxamide-2,2,2-trifluoroacetate;
N-(4-cyanobenzoyl)piperazine-1-carboxamide-2,2,2-trifluoroacetate;
N-(furan-2-carbonyl)piperazine-1-carboxamide-2,2,2-trifluoroacetate;
N-(4-nitrobenzoyl)piperazine-1-carboxamide-2,2,2-trifluoroacetate;
N-(2-naphthoyl)piperazine-1-carboxamide hydrochloride;
N-(4-cyanobenzoyl)piperazine-1-carboxamide hydrochloride;
N-(4-nitrobenzoyl)piperazine-1-carboxamide hydrochloride;
N-benzoylpiperazine-1-carboxamide hydrochloride;
N-(furan-2-carbonyl)piperazine-1-carboxamide hydrochloride;
N-(4-methylbenzoyl)piperazine-1-carboxamide hydrochloride;
N-(4-methoxybenzoyl)piperazine-1-carboxamide hydrochloride;
N-(4-chlorobenzoyl)piperazine-1-carboxamide hydrochloride;
N-(2,4-dichlorobenzoyl)piperazine-1-carboxamide-2,2,2-trifluoroacetate;
N-(4-bromobenzoyl)piperazine-1-carboxamide-2,2,2-trifluoroacetate;
N-(thiophene-2-carbonyl)piperazine-1-carbxamide-2,2,2-trifluoroacetate;
N-(benzo[b]thiophene-2-carbonyl)piperazine-1-carboxamide-2,2,2-trifluoroacetate;
N-(4-fluorobenzoyl)piperazine-1-carboxamide-2,2,2-trifluoroacetate;
N-isonicotinoylpiperazine-1-carboxamide-2,2,2-trifluoroacetate;
N-(2,4-dichlorobenzoyl)piperazine-1-carboxamide hydrochloride;
N-(4-bromobenzoyl)piperazine-1-carboxamide hydrochloride;
N-(benzo[b]thiophene-2-carbonyl)piperazine-1-carboxamide hydrochloride;
N-isonicotinoylpiperazine-1-carboxamide hydrochloride;
N-(4-fluorobenzoyl)piperazine-1-carboxamide hydrochloride;
N-(thiophene-2-carbonyl)piperazine-1-carboxamide hydrochloride;
N-(3-chlorobenzoyl)piperazine-1-carboxamide hydrochloride;
N-(3-methylbenzoyl)piperazine-1-carboxamide hydrochloride;
N-(3-methoxybenzoyl)piperazine-1-carboxamide hydrochloride;
N-(3-chlorobenzoyl)piperazine-1-carboxamide-2,2,2-trifluoroacetate;
N-(3-methylbenzoyl)piperazine-1-carboxamide-2,2,2-trifluoroacetate; and
N-(3-methoxybenzoyl)piperazine-1-carboxamide-2,2,2-trifluoroacetate.

6: A method for preventing or treating an emotional and behavioral disorder-related symptom, the method comprising administering to a subject in need thereof a pharmaceutical composition comprising the compound, the stereoisomer or a pharmaceutically acceptable salt thereof as defined in claim 1.

7: The method of claim 6, wherein the emotional and behavioral disorder-related symptom is any one selected from the group consisting of lack of sociability, lack of social cognitive intelligence, stereotyped behavior, hyperactivity, impulsivity distraction, and a combination thereof.

8: A method for preventing or treating an emotional and behavioral disorder, the method comprising administering to a subject in need thereof a pharmaceutical composition comprising the compound, the stereoisomer or the pharmaceutically acceptable salt thereof as defined in claim 1.

9: A method for preventing or treating a degenerative neurological disease, the method comprising administering to a subject in need thereof a pharmaceutical composition comprising the compound, the stereoisomer or the pharmaceutically acceptable salt thereof as defined in claim 1.

10: The method according to claim 8, wherein the emotional and behavioral disorder is any one selected from the group consisting of autism spectrum disorder, schizophrenia, obsessive-compulsive disorder, depression, anxiety disorder, panic disorder and attention deficit hyperactivity disorder.
